(19) United States
(12) Patent Application Publication
(10) Pub. No.: US 2019/0365266 A1
(43) Pub. Date: Dec. 5, 2019

(54) SIGNAL PROCESSING IN MAGNETOMETER FOR MEDICAL USE

(71) Applicant: CREAVO MEDICAL TECHNOLOGIES LIMITED, Leeds (GB)

(72) Inventors: Benjamin Thomas Hornsby VARCOE, Leeds (GB); David Diamante DIMAMBRO, Leeds (GB); Abbas Ahmad AL-SHIMARY, Leeds (GB); Richard Theodore GRANT, Leeds (GB)

(73) Assignee: Creavo Medical Technologies Limited, Leeds (GB)

(21) Appl. No.: 16/478,116

(22) PCT Filed: Aug. 3, 2018
(86) PCT No.: PCT/GB2018/052224

§ 371 Date: Jul. 15, 2019

(30) Foreign Application Priority Data
Aug. 18, 2017 (GB) 1713280.4

(51) Int. Cl. A61B 5/04; A61B 5/00

(52) U.S. Cl. CPC A61B 5/04007; A61B 5/7203; A61B 5/04012; A61B 5/7239

(57) ABSTRACT

A method of using a magnetometer system to analyse the magnetic field of a region of a subject's body is disclosed. The method comprises obtaining one or more signals corresponding to the time derivative of the time varying magnetic field of a region of a subject's body, averaging the time derivative signal or signals over plural periods, and using the averaged time derivative signal or signals to analyse the magnetic field generated by the region of the subject's body.

The present invention relates to a magnetometer for medical use, such as for use as a cardiac magnetometer.

It can be useful in many medical situations to be able to measure magnetic fields relating to or produced by the human body for diagnostic purposes. For example, the heart's magnetic field contains information that is not contained in an ECG (Electro-cardiogram), and so a magneto cardiogram scan can provide different and additional diagnostic information to a conventional ECG.

Most modern cardiac magnetometers are built using ultra-sensitive SQUID (Superconducting Quantum Interference Device) sensors. However, SQUID magnetometers are very expensive to operate as they require cryogenic cooling. Their associated apparatus and vacuum chambers are also bulky pieces of equipment. This limits the suitability of SQUID magnetometers for use in a medical environment, for example because of cost and portability considerations.

Another known form of magnetometer is an induction coil magnetometer. Induction coil magnetometers have the advantage over SQUID magnetometers that cryogenic cooling is not necessarily required, they are relatively inexpensive and easy to manufacture, they can be put to a wide range of applications and they have no DC sensitivity.

However, induction coil magnetometers have not been widely adopted for magneto cardiography because magneto cardiography requires low field (<nT), low frequency (<100 Hz) sensing, and common induction coil magnetometer designs that can achieve such sensitivities are too large to be practical for use as a cardiac probe.

The Applicants have addressed these problems in their earlier application WO2014/006387, which discloses a method and apparatus for detecting and analysing medically useful magnetic fields that uses an induction coil or coils of a specific configuration to detect the magnetic field of a subject.

Notwithstanding this, the Applicants believe that there remains scope for alternative arrangements and improvements to the design and use of magnetometers for medical use, and in particular for cardio magnetic sensing and/or imaging.

According to a first aspect of the present invention, there is provided a method of using a magnetometer system to analyse the magnetic field of a region of a subject's body, the method comprising:

obtaining one or more signals corresponding to the time derivative of the time varying magnetic field of a region of a subject's body;

averaging the time derivative signal or signals over plural periods; and

using the averaged time derivative signal or signals to analyse the magnetic field generated by the region of the subject's body.

According to a second aspect of the present invention, there is provided a magnetometer system for medical use, comprising:

one or more detectors for detecting the time varying magnetic field of a region of a subject's body;

detection circuitry configured to obtain from the one or more detectors one or more signals corresponding to the time derivative of a detected time varying magnetic field; and

averaging circuitry configured to average the time derivative signal or signals over plural periods;

wherein the magnetometer system is configured to use the averaged time derivative signal or signals to analyse the magnetic field generated by the region of the subject's body.

The present invention is concerned with a method of analysing the magnetic field of a region of a subject, such as their heart. In the present invention, one or more signals are obtained and averaged over plural periods, and then the averaged signal or signals are used to analyse the magnetic field generated by the region of the subject's body.

In contrast with conventional arrangements, however, the obtained signal or signals that are averaged and used to analyse the magnetic field of the region of the subject's body correspond to the time derivative of the magnetic field. As will be described further below, the Applicants have found that the use of the time derivative of the magnetic field in this manner can offer a number of advantages when compared with conventional techniques that use the magnetic field itself.

In particular, using the time derivative of the magnetic field can remove noise artefacts such as baseline wander from the signal, e.g. such that noise artefacts (e.g. baseline wander) can be removed from the signal without using filtering or while using relatively little filtering, and accordingly without affecting the “wanted” parts of the signal.

The Applicants have recognised in this regard that noise artefacts, such as baseline wander, can often themselves have a biological origin, and can therefore exhibit similar signal features to the “wanted” parts of the signal that have diagnostic importance. For example, movement of the body, e.g. limbs, of the subject can give rise to baseline wander in ECG signals, whereas a small shift in the baseline of the S-T segment of the ECG can indicate myocardial infarction. Similar effects can be observed in magneto-cardiogram (MCG) signals. As such, there is a risk when using filtering that “wanted” parts of the signal (e.g. that may have diagnostic importance) may be removed from the signal.

The Applicants have furthermore recognised that since the baseline wander is typically very low in frequency, its derivative is very small, and accordingly that using the time derivative of the magnetic field can effectively remove the baseline wander from the signal for analysis.

It will be appreciated therefore that the present invention provides an improved magnetometer system for medical use.

The magnetometer system of the present invention can be used as a system and probe to detect any desired magnetic field produced by a subject (by the human (or animal) body). It is preferably used to detect (and analyse) the time varying magnetic field of (or produced by) a region of the subject's body, such as their bladder, abdomen, chest or heart, head or brain, muscle(s), womb or one or more foetuses. Thus it may be, and is preferably, used to detect magnetic fields relating to the bladder, pregnancy, muscle activity, the brain, or the heart. In a preferred embodiment, the magnetometer is used for (and configured for) one or more of: magnetocardiography, magnetoencephalography, analysis and detection of bladder conditions (e.g. overactive bladder), analysis and detection of foetal abnormalities, and detection and analysis of pre-term labour.

In a particularly preferred embodiment the magnetometer is used as a cardiac magnetometer and to detect and analyse the magnetic field of a subject's heart.

Thus, according to another aspect of the present invention there is provided a method of analysing the magnetic field of a subject's heart, the method comprising:

obtaining one or more signals corresponding to the time derivative of the time varying magnetic field of a subject's heart;

averaging the time derivative signal or signals over plural periods; and

using the averaged time derivative signal or signals to analyse the magnetic field generated by the subject's heart.

According to another aspect of the present invention, there is provided a cardiac magnetometer system for analysing the magnetic field of a subject's heart, comprising:

one or more detectors for detecting the time varying magnetic field of a subject's heart;

detection circuitry configured to obtain from the one or more detectors one or more signals corresponding to the time derivative of a detected time varying magnetic field; and

averaging circuitry configured to average the time derivative signal or signals over plural periods;

wherein the magnetometer system is configured to use the averaged time derivative signal or signals to analyse the magnetic field generated by the subject's heart.

As will be appreciated by those skilled in the art, these aspects of the present invention can and preferably do include any one or more or all of the preferred and optional features of the invention described herein, as appropriate.

The one or more signals corresponding to the time derivative of the time varying magnetic field of the region of the subject's body may be obtained in any suitable manner and by any suitable device.

One or more detectors should be (and are preferably) used to obtain the signal(s) corresponding to the time derivative of the time varying magnetic field of the region of the subject's body. Thus, the magnetometer system of the present invention preferably comprises one or more detectors.

The magnetometer system of the present invention may comprise a single detector. In this case, the detector may be positioned appropriately over a subject (e.g. a subject's chest or other region of the subject's body) to take readings from a suitable (single) sampling position for the region of the subject's body in question. Alternatively, the detector may be moved over the subject (e.g. the subject's chest) to take readings from plural different sampling positions in use.

However, in one preferred embodiment, the magnetometer system comprises plural detectors, e.g. and preferably at least 7, e.g. 7-500 (or more), preferably at least 16, e.g. 16-500 (or more) detectors.

Where the magnetometer system comprises plural detectors, some or all of the detectors may be arranged in a two dimensional array, e.g. and preferably at least 7, preferably at least 16, detectors arranged in a two or three dimensional array. In this case, the or each detector array is preferably configured such that when positioned appropriately over a subject (e.g. a subject's chest or other region of the subject's body) the detector array can take readings from a suitable set of sampling positions without the need to further move the array over the subject.

The or each array can have any desired configuration, such as being a regular or irregular array, a hexagonal, rectangular or circular array (e.g. formed of concentric circles), etc.

The number and/or configuration of detectors in the or each array is preferably selected so as to provide an appropriate number of sampling points and/or an appropriate coverage for the region of the subject's body in question.

In a preferred embodiment, the detector array is configured to cover a region of biomagnetic interest, such as the torso or heart. In one such preferred embodiment, where the magnetometer is used as a cardiac magnetometer to detect and analyse the magnetic field of a subject's heart, the or each array comprises a hexagonal array of at least 7, e.g. 7-500 (or more), preferably at least 16, e.g. 16-500 (or more) detectors.

An increased number of detectors may be provided, e.g. where it is desired to measure the time-varying magnetic field of a subject's heart with a higher resolution and/or where it is desired to measure the time-varying magnetic field of a region of a subject's body other than the heart, such as in particular the brain. According to various preferred embodiments, the or each array may comprise a hexagonal array of 7, 19, 37, 61, 91, 127, 169, 217, 271, 331, 397 (or more) detectors.

The magnetometer system may comprise a single layer of detectors, or may comprise plural layers of one or more detectors, e.g. and preferably 2-10 (or more) layers, i.e. one above the other.

In one such embodiment, each detector layer comprises a single detector. In this case, then again, the magnetometer may be positioned appropriately over a subject (e.g. a subject's chest or other region of the subject's body) to take readings from a suitable (single) sampling position for the region of the subject's body in question. Alternatively, the magnetometer may be moved over the subject (e.g. the subject's chest) to take readings from plural different sampling positions in use. However, in a preferred embodiment, one or more or all of the detector layers comprise plural detectors, e.g. arranged in a two dimensional array, with one or more or each array preferably arranged as discussed above for the two dimensional array arrangement.

In these embodiments, one or more or each detector in each detector layer may be aligned with one or more or each detector in one or more or all of the other layers or otherwise (e.g. anti-aligned), as desired.

Where the magnetometer system comprises plural detectors, some or all of the detectors may be connected, e.g. in parallel and/or in series. Connecting plural detectors in series will have the effect of increasing the induced voltage for a given magnetic field strength. Connecting plural detectors in parallel will have the effect of reducing the thermal noise (Johnson noise) in the detectors. Preferably, a combination of series and parallel connections is used to optimise the balance of voltage and noise performance of the detectors.

In an embodiment, one or more or each detector in the magnetometer system is arranged in a gradiometer configuration, i.e. where two detectors are co-axially aligned (in the direction orthogonal to the plane in which each coil's windings are arranged), and where the signal from each of the coils is summed, e.g. to provide a measure of a change in the magnetic field in space.

The or each detector in the magnetometer system may comprise any suitable detector for detecting a time varying magnetic field.

The or each detector is preferably configured to be sensitive at least to magnetic signals between 0.1 Hz and 1 kHz, as this is the frequency range of the (majority of the) relevant magnetic signals of the heart. The or each detector may be sensitive magnetic signals outside of this range. The or each detector is preferably sensitive to magnetic fields in the range 10 fT-100 pT.

In the present invention, one or more signals corresponding to (indicative of) the time derivative of the time varying magnetic field of a region of a subject's body are obtained, averaged and used to analyse the magnetic field generated by the region of a subject's body. The one or more time derivative signals should (and preferably do) each comprise a signal having a time varying magnitude that corresponds to the time derivative of the time varying magnetic field of the region of the subject's body.

As such, the or each detector may be configured such that its output is a signal (e.g. current or voltage) corresponding to (having a time varying magnitude that corresponds to) the time derivative of the time varying magnetic field of the region of the subject's body. The output signal may then optionally be digitised. This represents a particularly convenient arrangement for obtaining a (e.g. digitised) signal corresponding to the time derivative of the time varying magnetic field of the region of the subject's body, since for example, it is not necessary to differentiate the “natural” signal produced by the detector. Indeed, in a preferred such embodiment, the signal (e.g. current or voltage) produced by the detector and/or the digitised signal is not (is other than) differentiated.

In a preferred such embodiment, one or more or each detector in the magnetometer system comprises an induction coil. Thus, an induction coil or coils (i.e. a coil that is joined to an amplifier at both ends) is preferably used to obtain (to detect) the signal or signals corresponding to the time derivative of the time varying magnetic field of the subject (e.g. of the subject's heart).

It should be noted here that the signal generated by an induction coil is the time derivative of the magnetic field. However, in conventional induction coil magnetometers, the output signal is immediately integrated over time to generate the wanted, useful signal. In contrast with this, in the present invention the time derivative signal is itself the wanted, useful signal, and so the output signal is preferably not (is other than) immediately integrated over time (and the (e.g. digitised) time derivative signal is instead averaged and used to analyse the magnetic field).

In these embodiments, each coil may be configured as desired.

Each coil preferably has a maximum outer diameter less than 10 cm, preferably less than 7 cm, preferably between 4 and 7 cm. By limiting the outer diameter of the coil to 10 cm or less, a coil having an overall size that can achieve a spatial resolution that is suitable for medical magnetometry (and in particular for magneto cardiography) is provided. In particular, this facilitates a medically applicable diagnostic using 16 to 50 (or more) sampling positions (detection channels) to generate an image. (As discussed above, and as will be appreciated by those skilled in the art, the data for each sampling position can, e.g., be collected either by using an array of coils, or by using one (or several) coils that are moved around the chest to collect the data.) In a preferred embodiment, coils of around 7 cm diameter are used.

One or more or each coil may have a non-magnetically active core (i.e. the coil windings may be wound around a non-magnetically active core), such as being air cored. Additionally or alternatively, one or more or each coil may have a magnetically active, such as ferrite or other magnetic material, core.

In one preferred embodiment, each coil corresponds to the arrangement described in the Applicants' earlier application WO2014/006387. Such coils can be used to provide a medical magnetometer that can be portable, relatively inexpensive, usable at room temperature and without the need for magnetic shielding, and yet can still provide sufficient sensitivity, accuracy and resolution to be medically useful. However, the or each coil need not comprise the optimised coil in accordance with WO2014/006387, and may have any suitable and desired configuration.

It will accordingly be appreciated that, in one preferred embodiment, the detector produces one or more time derivative signals, each comprising a voltage or current having a time varying magnitude that corresponds to the time derivative of the time varying magnetic field of the region of the subject's body. As such, in one preferred embodiment, obtaining one or more (e.g. digitised) signals corresponding to the time derivative of the time varying magnetic field of the region of the subject's body comprises using one or more detectors to produce a signal (e.g. current or voltage) having a time varying magnitude that corresponds to the time derivative of the time varying magnetic field of the region of the subject's body.

Each signal (e.g. current or voltage) from each detector may be digitised to produce a digitised signal having a time varying magnitude that corresponds to the time derivative of the time varying magnetic field of the region of the subject's body.

Thus, in a preferred embodiment, the “raw” signal or signals (e.g. current or voltage) generated by the one or detectors are digitised, e.g. using one or more digitisers.

In these embodiments, the or each digitiser may comprise any suitable digitiser that is operable to digitise (convert) an analogue signal received from the one or more detectors into a digital signal, e.g. for further processing. The digitiser should (and preferably does) convert a voltage or current generated in the one or more detectors by the magnetic field into a digital signal.

In a preferred embodiment, the magnetometer system comprises a digitiser coupled to each detector (each coil) and configured to digitise a signal from the detector. Where the system includes plural detectors, each detector may have its own, respective and separate, digitiser (i.e. there will be as many digitisers as there are detectors), or some or all of the detectors may share a digitiser.

In a preferred embodiment, the or each digitiser comprises an analogue to digital converter (ADC).

The or each digitiser may be directly connected to the or each respective detector, or more preferably, the or each digitiser may be connected to the or each respective detector via an amplifier. Thus in a preferred embodiment, the magnetometer system includes one or more detection amplifiers, preferably in the form of a microphone amplifier (a low impedance amplifier), connected to one or more or each detector, e.g. to the ends of each coil. The or each detection amplifier is preferably then connected to a digitiser or digitisers.

The or each amplifier may be configured to have any suitable and desired amplification level. The or each amplifier may, for example, amplify the signal (including the noise) received from the or each detector by around 1000 times (60 dB) or more.

In a preferred embodiment, the magnetometer system is arranged such that the detector (e.g. coil) and amplifier (that is coupled to the detector (coil)) are arranged together in a sensor head or probe which is then joined by a wire to the remaining components of the magnetometer system to allow the sensor head (probe) to be spaced from the remainder of the magnetometer system in use.

It will accordingly be appreciated that, in one preferred embodiment, obtaining one or more (e.g. digitised) signals corresponding to the time derivative of the time varying magnetic field of the region of the subject's body comprises using one or more detectors to detect the time derivative of the time varying magnetic field of the region of the subject's body, and preferably digitising the signal or signals (e.g. voltage or current) output from the one or more detectors to produce a digitised signal or signals having a time varying magnitude that corresponds to the time derivative of the time varying magnetic field of the region of the subject's body.

Although it is particularly preferred for the or each detector to be configured such that its output is a signal corresponding to the time derivative of the time varying magnetic field, it would also or instead be possible to use one or more detectors configured such that its output is a signal (e.g. current or voltage) corresponding to (indicative of) the time varying magnetic field of the region of the subject's body. That is, the or each detector may be configured such that its output is a signal (e.g. current or voltage) having a time varying magnitude that corresponds to the time varying magnetic field of the region of the subject's body. In these embodiments, the (e.g. digitised) signal should be (and is preferably) differentiated to obtain a (e.g. digitised) signal corresponding to the time derivative of the time varying magnetic field of the region of the subject's body.

Thus, in a preferred embodiment, obtaining one or more signals corresponding to the time derivative of the time varying magnetic field of the region of the subject's body comprises using one or more detectors to detect the time varying magnetic field of the region of the subject's body, optionally digitising the signal or signals (e.g. voltage or current) output from the one or more detectors to produce a digitised signal or signals having a time varying magnitude that corresponds to the time varying magnetic field of the region of the subject's body, and differentiating the (e.g. digitised) signal or signals to obtain the one or more (e.g. digitised) signals corresponding to (having a time varying magnitude that corresponds to) the time derivative of the time varying magnetic field of a region of a subject's body.

In this regard, the Applicants have found that the above described benefits associated with the use of the derivative (i.e. the removal of noise artefacts such as baseline wander) can still be obtained when using a detector whose output signal corresponds to the time varying magnetic field, i.e. by differentiating the output signal to obtain a signal corresponding to the time derivative of the time varying magnetic field (and then averaging the time derivative signal and using the averaged time derivative signal to analyse the magnetic field as described above).

In these embodiments, the detector or detectors may each comprise any suitable detector, such as, for example, a SQUID (Superconducting Quantum Interference Device) sensor, a flux gate magnetometer, a tunnelling magneto resistive (TMR) sensor, an Atomic Physics Magnetometer, etc.

In these embodiments, the differentiation may be performed in any suitable manner. Where, for example, the (digitised) signal comprises a sequence of values,

V(t)=[V₁,V₂,V₃, . . . ,Vₙ],

and where the values Vᵢ, Vᵢ₊₁ are separated by a fixed time step δt, then the derivative may be approximated by:

$$\frac{dV}{dt} \approx [ \frac{{V}_{1} - {V}_{2}}{δ t} , \frac{{V}_{2} - {V}_{3}}{δ t} , \frac{{V}_{3} - {V}_{4}}{δ t} , … , \frac{{V}_{n - 1} - {V}_{n}}{δ t} ] .$$

In the present invention, the (e.g. digitised) time derivative signal or signals is averaged over plural periods, e.g. using averaging circuitry (e.g. in the form of hardware or software). The averaging should be (and is preferably) performed on a signal or signals in the time derivative domain, i.e. on the time derivative signal itself (i.e. without, e.g., firstly integrating the (e.g. digitised) time derivative signal). The averaged (e.g. digitised) time derivative signal or signals should (and preferably do) each have a magnitude that corresponds to the averaged time derivative of the time varying magnetic field of the region of the subject's body.

The (e.g. digitised) time derivative signal or signals may be averaged over plural periods as desired, and the averaging circuitry may comprise any suitable and desired circuitry for averaging the time derivative signal or signals over plural periods.

In a preferred embodiment, the time derivative signal or signals, e.g. received from the detector or detectors (or from the digitiser or digitisers), are averaged over plural periods, i.e. over plural cycles of the periodic (or pseudo-periodic) signal.

In an embodiment, a trigger is provided and used for gating (windowing) the time derivative signal (i.e. for identifying and dividing the periodic (or pseudo-periodic) signal into its plural repeating periods). The trigger should be, and preferably is, synchronised with the time varying magnetic field of the region of the subject's body. For example, where the magnetometer is used to analyse the magnetic field of a subject's heart, then the signal is preferably averaged over a number of heartbeats, and an ECG or Pulse Ox trigger from the test subject may be used as a detection trigger for the signal acquisition process.

Thus, in a preferred embodiment, a trigger is used to identify each repeating period of the periodic (or pseudo-periodic) time derivative signal, and then the signal is averaged over the plural identified periods. Thus, in a preferred embodiment, plural repeating periods of the derivative of the time varying magnetic field of a region of a subject's body are detected, (preferably digitised) and averaged overall plural periods.

In a preferred embodiment, the trigger is determined based on (using) the shape of a signal (waveform) and/or a threshold detection. In a particularly preferred such embodiment, the trigger is determined based on (using) the shape of a time derivative signal (waveform) and/or a threshold detection using the time derivative signal.

In this regard, the Applicants have recognised that the use of a trigger derived from an ECG or MCG signal itself can be prone to errors, e.g. due to noise artefacts such as baseline wander. In contrast, since as described above, the use of the time derivative signal can remove noise artefacts such as baseline wander, the use of a time derivative signal to determine the trigger has the effect of improving the reliability of the triggering.

Thus, in a particularly preferred embodiment, a time derivative signal (e.g. a signal corresponding to the time derivative of the time varying magnetic field or a signal corresponding to the time derivative of the time varying electric potential of a region of a subject's body) is used to determine a detection trigger for the signal acquisition process.

Other arrangements would be possible. For example, each repeating period of the (periodic) signal may be identified without the use of a trigger, and then the signal may be averaged over the plural identified periods.

Once the (e.g. digitised) time derivative signal or signals have been averaged over plural periods, then the averaged time derivative signal or signals may (or may not) be subjected to further processing, i.e. before being used to analyse the magnetic field generated by the region of the subject's body.

In a preferred embodiment, the time derivative signal or signals is subjected to further processing, i.e. before being used to analyse the magnetic field generated by the region of the subject's body.

In a preferred such embodiment, the (e.g. digitised) time derivative signal or signals is filtered (before the averaged signal or signals is used to analyse the magnetic field generated by the region of the subject's body). In this case, the time derivative signal or signals may be filtered in any suitable manner.

In a preferred embodiment, the (e.g. digitised) time derivative signal or signals is filtered using a filter or filters, wherein the filter or filters are configured to attenuate (e.g. to remove) (at least some) environmental noise in the signal or signals.

The time derivative signal or signals may be filtered to attenuate (e.g. to remove) (at least some) environmental noise such as magnetic noise from power lines and other environmental noise sources (e.g. elevators, air conditioners, nearby traffic, mechanical vibrations).

It would be possible to perform the filtering before signal averaging. Thus, in one embodiment, the time derivative signal or signals is filtered (and the time derivative signal or signals that is averaged comprises the filtered signal or signals). However, in a preferred embodiment, the filtering is performed after signal averaging.

Thus, the method may further comprise filtering (and the system may comprise a filter configured to filter) the averaged time derivative signal or signals, i.e. using a filter or filters.

The filter or filters should be (and are preferably) configured to filter the time derivative signal or signals so as to produce a filtered time derivative signal or signals.

In one embodiment, the attenuated part of the (e.g. digitised) time derivative signal or signals is discarded (i.e. not used). Thus, in an embodiment, the filter or filters is configured to filter the time derivative signal or signals so as to remove (and discard) the environmental noise.

However, it would also be possible to retain the environmental noise (the attenuated (removed)) part of the time derivative signal or signals, and to use it for some other purpose. Thus, in an embodiment, the filter or filters is configured to filter the time derivative signal or signals so as to produce both (e.g. to separate out) the filtered time derivative signal or signals and one or more other (e.g. environmental noise) signals.

The filter or filters may be configured to attenuate environmental noise in the time derivative signal or signals, i.e. so as to produce the filtered time derivative signal or signals. In this regard, attenuating the environmental noise should (and preferably does) comprise reducing the amplitude of the environmental noise (e.g. at least in the filtered time derivative signal or signals). More preferably, attenuating the environmental noise comprises (completely) removing the environmental noise (e.g. at least from the filtered time derivative signal or signals).

The filter or filters should be (and is preferably) configured to attenuate (e.g. separate or remove) the environmental noise in the time derivative signal or signals without attenuating (or attenuating to a lesser degree), and preferably without (significantly) distorting, some or all of the “useful”, wanted, part of the time derivative signal.

In this regard, the conventional approach to analysing the magnetic field of a subject's heart is to keep as much of the signal originating from the heart as possible. This will include the P wave, the QRS wave and/or the T wave. Thus, conventionally, care is taken to retain as much of the P wave, the QRS wave and the T wave in the signal as possible. The Applicants have found that environmental noise can appear in a frequency range that overlaps with the frequency range of this conventionally “wanted” signal.

However, the Applicants have furthermore recognised that the QRS complex is particularly important in terms of providing diagnostic information, and that the T-wave is less important in this regard. The Applicants have also recognised that environmental noise can appear (mainly) in a frequency range that overlaps with the frequency range of the T-wave. This means that the filter can be (and is preferably) configured to attenuate (e.g. separate or remove) the environmental noise (together with the T-wave) in the time derivative signal or signals without attenuating (or attenuating to a lesser degree), and preferably without (significantly) distorting, the “useful”, wanted, QRS complex.

Thus, the filter or filters is preferably configured to allow at least the QRS complex to pass (preferably without being attenuated and/or distorted) and to attenuate (e.g. to separate or remove) environmental noise, i.e. so as to produce the filtered time derivative signal or signals. Filtering the time derivative signal or signals in this manner allows environmental noise to be removed from the signal, without (significantly) affecting the medically useful QRS complex.

In this regard, the Applicants have recognised that environmental noise can comprise (mainly) lower frequency components, e.g. when compared with the frequency range at which the QRS complex appears. Thus, the filter is preferably configured to allow at least the QRS complex to pass (preferably without being attenuated and/or distorted) and to attenuate (e.g. to separate or remove) parts of the time derivative signal having frequencies less than the frequency range at which the QRS complex appears.

In a preferred embodiment, the filter is configured to attenuate (e.g. to separate or remove) time derivative signal or signals having frequencies below a particular, preferably selected, cut-off frequency (threshold) (i.e. the filter is configured to attenuate components of the time derivative signal or signals with frequencies below the cut-off frequency). The filter may be configured to attenuate (e.g. to separate or remove) only some frequencies less than the cut-off frequency, but more preferably the filter is configured to attenuate (e.g. to separate or remove) all frequencies less than the cut-off frequency.

Thus, in a preferred embodiment, the or each filter comprises a high-pass filter, i.e. where the high-pass filter has a low frequency cut-off (i.e. a frequency (threshold) below which (most of) the time derivative signal is attenuated (but above which (most of) the time derivative signal is passed by the high-pass filter)), and filtering the time derivative signal or signals comprises high-pass filtering the time derivative signal or signals.

The or each high-pass filter may be configured in any suitable manner. In a particularly preferred embodiment, the high-pass filter comprises a windowed sinc filter. This is a particularly beneficial arrangement since the windowed sinc filter can provide a good approximation to the ideal “brick wall” high-pass filter.

The low frequency cut-off may be selected as desired. However, in a preferred embodiment, the filter has a low frequency cut-off between around 8 and 12 Hz, more preferably between around 9 and 11 Hz. Most preferably, the filter is configured to have a low frequency cut-off at around 10 Hz.

In this regard, the Applicants have found in particular that environmental noise can appear in the frequency range around <10 Hz, whereas the T-wave appears in the frequency range around 4-7 Hz and the QRS complex appears at frequencies >10 Hz. Accordingly, the use of a low frequency cut-off at around 10 Hz can result in removal of a significant proportion of environmental noise from the time derivative signal or signals, without significantly affecting the medically useful part of the time derivative signal or signals.

The filter or filters is preferably configured to have a relatively narrow roll-off. Again, this means that the filter will function as close as possible to the ideal “brick wall” filter.

In this regard, the Applicants have recognised that configuring the filter in this manner will have the effect of increasing the pass band and/or stop band ripple, but that the shape of the roll off is more important, where it is desired to remove environmental noise from the time derivative signal. This is because the environmental noise can appear adjacent in frequency to the useful QRS complex part of the time derivative signal.

In a particularly preferred embodiment, the filter or filters is additionally configured to attenuate (e.g. to separate or remove) other (high-frequency) background noise in the time derivative signal or signals. As such, a single filter may be (and is preferably) used to attenuate multiple types of noise in the time derivative signal or signals.

In these embodiments, the or each filter should be (and is preferably) configured to attenuate the other (high-frequency) background noise in the time derivative signal or signals without attenuating (or attenuating to a lesser degree), and preferably without (significantly) distorting, at least some of the “useful”, wanted, part of the signal. Thus, the filter is preferably configured to allow at least the QRS complex to pass (preferably without being attenuated and/or distorted) and to attenuate (e.g. to separate or remove) the other (high-frequency) background noise.

In this regard, the Applicants have recognised that other background noise that has (mainly) relatively high frequency components (e.g. when compared with the frequency range at which the QRS complex appears), such as mains power noise, may be present in the time derivative signal or signals. Thus, the filter is preferably configured to allow at least the QRS complex to pass (preferably without being attenuated and/or distorted) and to attenuate (e.g. to separate or remove) parts of the time derivative signal having frequencies greater than the frequency range at which the QRS complex appears.

In a preferred embodiment, the filter or filters is configured to attenuate (e.g. to separate or remove) time derivative signal or signals having frequencies higher than a particular, preferably selected, high frequency cut-off frequency (threshold) (i.e. the filter is configured to attenuate components of the time derivative signal or signals with frequencies above the high frequency cut-off frequency). The filter may be configured to attenuate only some frequencies higher than the high frequency cut-off frequency, but more preferably the filter is configured to attenuate all frequencies higher than the high frequency cut-off frequency.

Thus, in a preferred embodiment, the filter or filters comprises a low-pass filter, i.e. where the low-pass filter has a high frequency cut-off (i.e. a frequency (threshold) above which (most of) the time derivative signal is attenuated (but below which (most of) the time derivative signal is passed by the low-pass filter)), and filtering the time derivative signal or signals comprises low-pass filtering the time derivative signal or signals.

The low-pass filter may be configured in any suitable manner. In a particularly preferred embodiment, the low-pass filter comprises a windowed sinc filter.

The high frequency cut-off may be selected as desired.

In this regard, the Applicants have found, in particular that the other (high-frequency) background noise, in particular environmental noise such as mains power noise, appears in the frequency range around 50 Hz, whereas the QRS complex appears at frequencies <50 Hz, and accordingly that the use of a high frequency cut-off at around 50 Hz (and preferably less than this) results in removal of a significant proportion of the other (high-frequency) background noise from the time derivative signal or signals, without significantly affecting the medically useful part of the time derivative signal or signals.

Thus, in a preferred embodiment, the filter has a high frequency cut-off at or below around 50 Hz, preferably between around 45 and 50 Hz, more preferably between around 45 and 48 Hz.

Where the mains power noise appears at another frequency, e.g. at around 60 Hz, then the filter may be configured to have a high frequency cut-off at or below that other frequency. Thus, in a preferred embodiment, the filter has a high frequency cut-off at or below around 60 Hz, preferably between around 55 and 60 Hz, more preferably between around 55 and 58 Hz.

It will accordingly be appreciated that in a particularly preferred embodiment, the filter is configured to attenuate (e.g. to separate or remove) environmental noise and other (high-frequency) background noise in the time derivative signal or signals, preferably without attenuating (or attenuating to a lesser degree), and preferably without (significantly) distorting, the “useful”, wanted, part of the time derivative signal, i.e. the QRS complex.

In a preferred embodiment, the filter is configured to allow at least the QRS complex to pass (preferably without being attenuated and/or distorted) and to attenuate (e.g. to separate or remove) parts of the time derivative signal having frequencies outside the frequency range at which the QRS complex appears.

In a preferred embodiment, the filter or filters is configured to attenuate (e.g. to separate or remove) time derivative signal or signals having frequencies below a particular, preferably selected, low frequency cut-off (threshold) and to attenuate (e.g. to separate or remove) time derivative signal or signals having frequencies above a particular, preferably selected, high frequency cut-off (threshold). Thus, the filter or filters is preferably configured to attenuate time derivative signal or signals having frequencies outside a particular, preferably selected, frequency range.

The filter may be configured to attenuate (e.g. to separate or remove) only some frequencies higher than the high frequency cut-off and only some frequencies less than the low frequency cut-off, but more preferably the filter is configured to attenuate (e.g. to separate or remove) all frequencies higher than the high frequency cut-off and all frequencies less than the low frequency cut-off.

Thus, in a preferred embodiment, the filter or filters comprises a band-pass filter, i.e. where the band-pass filter has a low frequency cut-off (threshold) and a high frequency cut-off (threshold), and filtering the time derivative signal or signals comprises band-pass filtering the time derivative signal or signals, i.e. so as to produce the filtered time derivative signal or signals.

The or each band-pass filter may be configured in any suitable manner. In a particularly preferred embodiment, the band-pass filter comprises a combination of (i.e. the difference between) two windowed sinc filters.

The windowed sinc filter or filters should be (and preferably are) configured to have a particular, preferably selected, window function. The filter window function or functions may be selected as desired. Suitable window functions include, for example, the Hamming window, the Blackman window, the Bartlett window, the Hanning window, etc.

In a particularly preferred embodiment, the or each windowed sinc filter uses a Blackman window. The Applicants have found that the Blackman window is particularly suited for use in preferred embodiments of the present invention. Although the Blackman window has a slower roll-off compared with the other types of window function (e.g. the Hamming window), it has an improved stopband attenuation, and a lower passband ripple.

Similarly, the or each windowed sinc filter should (and preferably does) have a particular, preferably selected, filter kernel length, M. In the frequency domain, the length of the filter kernel M determines the transition bandwidth of the filter, BW. There is a trade-off between computation time (which depends on the value of M) and the filter sharpness (the value of BW), which can be expressed through the approximation:

$$M \approx \frac{4}{BW} .$$

As such, the sharper the filter is (the smaller the transition bandwidth BW), the longer is the time required to perform convolution in the time domain.

The filter is preferably configured to have a relatively narrow roll-off. Again, this means that the filter will function as close as possible to the ideal “brick wall” filter.

In a particularly preferred embodiment, the length of the filter kernel, M is set to be equal to one second, i.e. of averaged signal (and therefore to be equal to the sampling rate). This minimises the transition bandwidth BW.

The passband of the band pass filter may be selected as desired. However, in a preferred embodiment, the passband has a low frequency cut-off between around 8 and 12 Hz, and a high frequency cut-off between around 45 and 50 Hz, more preferably between around 45 and 48 Hz. It would also be possible for the high frequency cut-off to be between around 55 and 60 Hz, more preferably between around 55 and 58 Hz, e.g. as described above. Most preferably, the filter is configured to have a passband at around 10 to 50 Hz.

The Applicants have found that this arrangement provides a practical and efficient way to examine the signal and extract the “useful” MCG features reliably, especially in a noisy environment. However, other arrangements would be possible.

The averaged (e.g. digitised) time derivative signal or signals may be subjected to other types of processing, i.e. before being used to analyse the magnetic field generated by the region of the subject's body, if desired.

In the present invention, the averaged time derivative signal or signals (i.e. that each have a magnitude that corresponds to the averaged time derivative of the time varying magnetic field of the region of the subject's body) is used to analyse the magnetic field generated by the region of the subject's body. That is, an averaged signal that is in the time derivative domain (and not in the time domain (integrated time domain)) is used to analyse the magnetic field generated by the region of the subject's body.

In the present invention, the time derivative signal or signals should be (and is preferably) retained in the time derivative domain, i.e. for use in analysing the magnetic field generated by the region of the subject's body. Preferably, at no point is the time derivative signal or signals (nor the averaged time derivative signal or signals) converted from the derivative domain to the time domain (i.e. at no point is the time derivative signal or signals nor the averaged time derivative signal or signals integrated).

The averaged signal that is in the time derivative domain (and not in the time domain (integrated time domain)) may be used to analyse the magnetic field generated by the region of the subject's body in any suitable manner (without integrating the averaged time derivative signal or signals).

A heartbeat's waveform and/or information such as a time interval or intervals e.g. between separate heartbeats and/or between certain features within a single heartbeat, and/or a shape or shapes of a heartbeat(s) may be obtained from the time derivative signal or signals.

In one preferred embodiment, the averaged signal or signals are subjected to appropriate signal processing (without integrating), for example to generate false colour images, a heat map, and/or a spatial topographic image of the derivative of the magnetic field or otherwise.

Thus, in a preferred embodiment, the averaged (e.g. digitised) time derivative signal or signals are used to provide an output indicative of the derivative of the time varying magnetic field (and not (other than) indicative of the magnetic field). This preferably comprises providing a display indicative of the derivative of the time varying magnetic field (and not (other than) indicative of the magnetic field), e.g. displaying an image indicative of the derivative of the time varying magnetic field on a display. Most preferably, the averaged signal or signals are used to provide a false colour image or images indicative of the derivative of the time varying magnetic field (and not (other than) indicative of the magnetic field), and the false colour image or images are displayed on a display.

In a preferred embodiment, suitable measurements are taken to allow an appropriate magnetic scan image of the heart (or other body region of interest) to be generated, which image can then, e.g., be compared to reference images for diagnosis. The present invention can be used to carry out any known and suitable procedure for imaging the magnetic field of the heart.

Preferably 7 to 500 (or more) (e.g. as described above) sampling positions (detection channels) are detected in order to generate the desired scan image.

Additionally or alternatively, one or more diagnostic parameters may be (e.g. automatically) extracted from the (optionally processed) averaged (e.g. digitised) time derivative signal or signals (without integration).

Thus, in a preferred embodiment, using the averaged time derivative signal or signals to analyse the magnetic field generated by the region of the subject's body comprises extracting one or more diagnostic parameters from the averaged time derivative signal or signals (and not from the magnetic field) (and without integrating).

Extracting one or more diagnostic parameters may comprise determining a height, width, amplitude, slope, gradient, rate of change, shape and/or area from one or more regions of the averaged digitised time derivative signal or signals (without integration). The height, width, amplitude, slope, gradient, rate of change, shape, or area may be a height, width, amplitude, slope, gradient, rate of change, shape or area of a signal feature in the averaged time derivative signal or signals.

For example, the height, width, amplitude, slope, gradient, rate of change, shape and/or area of the repeating P-P interval, P-wave, P-R (or P-Q) segment, P-R (or P-Q) interval, QRS complex, S-T segment, T-wave, S-T interval, Q-T interval, and/or T-P segment, etc., may be extracted from the averaged time derivative signal or signals (without integration).

It should be noted that when analysing the magnetic field in the derivative domain, the rate of change, gradient, or slope of a feature may be used. The gradient of a feature in the integral corresponds to the amplitude of a feature in the derivative. This can allow more detailed or accurate diagnostic information to be obtained.

For example, in the “normal” time domain ECG (and in the “normal” time domain MCG) the QRS complex comprises a single peak. It can be challenging to determine (or accurately measure), e.g., a slight imbalance or asymmetry in the QRS peak, e.g. if one side of the ECG QRS peaks falls faster or slower than it rises (or vice versa).

By contrast, when using the derivative (MCG or ECG) signal, the QRS complex comprises two peaks, one corresponding to the rising edge “QR” and one to the falling edge, the “RS”, of the “normal” time domain QRS complex. This means that, when using the derivative domain, any difference (e.g. imbalance or asymmetry) as described above is much easier to detect, e.g. since the two peaks will have different shapes and/or amplitudes. The same is true for other peaks and signal features in the averaged time derivative signal or signals.

In addition, small fluctuations on large absolute values (e.g. signals with large offsets or DC biases) can more readily be seen when using the derivative compared to when using the integral. This is because upwards or downwards trends (or gradients/slopes) can be seen as positive or negative features in the derivative. For a sufficiently offset (or biased) signal, all values may remain positive (or negative) despite small fluctuations making it difficult to establish a trend.

As such, using the derivative domain in the manner of various embodiments can make diagnostic measurements more resistant to offsets or (e.g. DC) biases, i.e. since only change is measured. This can make it easier to deal with situations, for example, where a threshold value is of interest and is required to be measured. In particular, this can address the situation where, for example, it is desired to determine the value or location of a change from a positive to negative value in the MCG signal, but where because of an offset or (e.g. DC) bias, all values of the signal are positive or negative.

The one or more diagnostic parameters may be compared to reference parameters for diagnosis, if desired.

The present invention accordingly extends to the use of the magnetometer system of the present invention for analysing, e.g. imaging and/or extracting one or more diagnostic parameters from, the magnetic field generated by a subject's heart (or other body region), and to a method of analysing, e.g. imaging and/or extracting one or more diagnostic parameters from, the magnetic field generated by a subject's heart (or other body region) comprising using the method or system of the present invention to analyse, e.g. to image and/or extract one or more diagnostic parameters from, the magnetic field generated by a subject's heart (or other region of the body). The analysis, and preferably the generated image and/or one or more diagnostic parameters, is preferably used for diagnosis of (to diagnose) a medical condition, such as abnormality of the heart, etc.

Thus according to another aspect of the present invention, there is provided a method of diagnosing a medical condition, comprising:

obtaining one or more signals corresponding to the time derivative of the time varying magnetic field of a region of a subject's body;

averaging the time derivative signal or signals over plural periods;

using the averaged time derivative signal or signals to analyse the magnetic field generated by the region of the subject's body; and

using the analysis of the magnetic field generated by the region of the subject's body to diagnose said medical condition.

In this aspect of the present invention, the signal (features of interest) from the detector or detectors are preferably used to produce an image representative of the (time derivative of the) magnetic field generated by the region of the subject's body and/or to extract one or more diagnostic parameters, and the method preferably then comprises comparing the image and/or the one or more diagnostic parameters obtained with a reference image or images and/or parameter or parameters to diagnose the medical condition. The medical condition is, as discussed above, preferably one of: abnormality of the heart, a bladder condition, pre-term labour, foetal abnormalities or abnormality of the head or brain.

As will be appreciated by those skilled in the art, these aspects and embodiments of the present invention can and preferably do include any one or more or all of the preferred and optional features of the invention described herein, as appropriate.

Although as described above, the use of the time derivative signal according to the present invention is particularly beneficial for analysing the magnetic field of a region of a subject's body, it is also useful for analysing the electric potential of a region of a subject's body, i.e. for ECG measurements.

Thus, according to a third aspect of the present invention, there is provided a method of using an electrocardiography system to analyse the electric potential of a region of a subject's body, the method comprising:

obtaining one or more signals corresponding to the time derivative of the time varying electric potential of a region of a subject's body;

averaging the time derivative signal or signals over plural periods; and

using the averaged time derivative signal or signals to analyse the electric potential generated by the region of the subject's body.

According to a fourth aspect of the present invention, there is provided a electrocardiography system for medical use, comprising:

one or more detectors for detecting the time varying electric potential of a region of a subject's body;

detection circuitry configured to obtain from the one or more detectors one or more signals corresponding to the time derivative of a detected time varying electric potential; and

averaging circuitry configured to average the time derivative signal or signals over plural periods;

wherein the electrocardiography system is configured to use the averaged time derivative signal or signals to analyse the electric potential generated by the region of the subject's body.

As will be appreciated by those skilled in the art, these aspects of the invention can and preferably do include any one or more or all of the preferred and optional features of the present invention, as appropriate. In particular, where appropriate, any one or more or all of the preferred and optional features described above in terms of the magnetic field may be adapted in terms of the electric potential and included in these aspects.

Thus, one or more (e.g. digitised) signals corresponding to (indicative of) the time derivative of the time varying electric potential of a region of a subject's body may be obtained, averaged and used to analyse the electric potential generated by the region of a subject's body. The one or more (e.g. digitised) time derivative signals should (and preferably do) each comprise a signal having a time varying magnitude that corresponds to the time derivative of the time varying electric potential of the region of the subject's body.

In these aspects and embodiments, one or more detectors are preferably used to produce a signal having a time varying magnitude that corresponds to the time derivative of the time varying electric potential of the region of the subject's body, and that signal may optionally be digitised, e.g. and preferably as described above. Additionally or alternatively, one or more detectors may be used to produce a signal having a time varying magnitude that corresponds the time varying electric potential of the region of the subject's body, and then the electric potential signal may be (optionally digitised and) differentiated to obtain the one or more signals corresponding to the time derivative of the time varying electric potential of the region of the subject's body, e.g. and preferably as described above.

As will be appreciated from the above, a particular advantage of the present invention is that it can be used in the normal hospital or surgery or other environment, without the need for (external) magnetic shielding. Thus, in a particularly preferred embodiment, the methods of the present invention comprise using the magnetometer system to detect the magnetic field of a subject's heart (or other body region) in a non-magnetically shielded environment (and without the use of (external) magnetic shielding). (It would, however, be possible to use the magnetometer system to detect the magnetic field of a subject's heart (or other body region) in a magnetically shielded environment (and with the use of (external) magnetic shielding), if desired.)

It should be noted that, as used herein, a “magnetically shielded environment” is intended to include arrangements where a magnetometer is either arranged in a shielded room or enclosure. In such arrangements, both the subject being measured and the magnetometer are contained within the same shielded room or enclosure. By contrast, as used herein, a magnetometer may be considered to be in a “non-magnetically shielded environment” where no external piece or pieces of apparatus are used to protect the subject being measured, nor the magnetometer doing the measuring.

Correspondingly, a particular advantage of the present invention is that it can be used without the need for cooling such a cryogenic cooling. Thus, in a particularly preferred embodiment, the methods of the present invention comprise using the magnetometer system to detect the magnetic field of a subject's heart (or other body region) without the use of (e.g. cryogenic) cooling. (It would, however, be possible to use the magnetometer system to detect the magnetic field of a subject's heart (or other body region) with the use of (e.g. cryogenic) cooling, if desired.)

As will be appreciated by those skilled in the art, all of the aspects and embodiments of the invention described herein can and preferably do include any one or more or all of the preferred and optional features of the present invention, as appropriate.

Any one or more or all of the processing circuitry described herein (such as in particular the detection circuitry, the averaging circuitry, and/or the processing circuitry) may be embodied in the form of one or more fixed-function units (hardware), and/or in the form of programmable processing circuitry (hardware) that can be programmed to perform the desired operation, and/or in the form of software e.g. computer program(s). Equally, any one or more of the processing circuitry described herein may be provided as a separate circuit element to any one or more of the other processing circuitry, and/or any one or more or all of the processing circuitry may be at least partially formed of shared processing circuitry.

The methods in accordance with the present invention may be implemented at least partially using software e.g. computer programs. It will thus be seen that when viewed from further aspects the present invention provides computer software specifically adapted to carry out the methods herein described when installed on data processing means, a computer program element comprising computer software code portions for performing the methods herein described when the program element is run on data processing means, and a computer program comprising code means adapted to perform all the steps of a method or of the methods herein described when the program is run on a data processing system. The data processing system may be a microprocessor, a programmable FPGA (Field Programmable Gate Array), etc.

The invention also extends to a computer software carrier comprising such software which when used to operate a magnetometer system comprising data processing means causes in conjunction with said data processing means said system to carry out the steps of the methods of the present invention. Such a computer software carrier could be a physical storage medium such as a ROM chip, CD ROM or disk, or could be a signal such as an electronic signal over wires, an optical signal or a radio signal such as to a satellite or the like.

It will further be appreciated that not all steps of the methods of the invention need be carried out by computer software and thus from a further broad aspect the present invention provides computer software and such software installed on a computer software carrier for carrying out at least one of the steps of the methods set out herein.

The present invention may accordingly suitably be embodied as a computer program product for use with a computer system. Such an implementation may comprise a series of computer readable instructions either fixed on a tangible medium, such as a non-transitory computer readable medium, for example, diskette, CD ROM, ROM, or hard disk. It could also comprise a series of computer readable instructions transmittable to a computer system, via a modem or other interface device, over either a tangible medium, including but not limited to optical or analogue communications lines, or intangibly using wireless techniques, including but not limited to microwave, infrared or other transmission techniques. The series of computer readable instructions embodies all or part of the functionality previously described herein.

Those skilled in the art will appreciate that such computer readable instructions can be written in a number of programming languages for use with many computer architectures or operating systems. Further, such instructions may be stored using any memory technology, present or future, including but not limited to, semiconductor, magnetic, or optical, or transmitted using any communications technology, present or future, including but not limited to optical, infrared, or microwave. It is contemplated that such a computer program product may be distributed as a removable medium with accompanying printed or electronic documentation, for example, shrink wrapped software, pre-loaded with a computer system, for example, on a system ROM or fixed disk, or distributed from a server or electronic bulletin board over a network, for example, the Internet or World Wide Web.

A number of preferred embodiments of the present invention will now be described by way of example only and with reference to the accompanying drawings, in which:

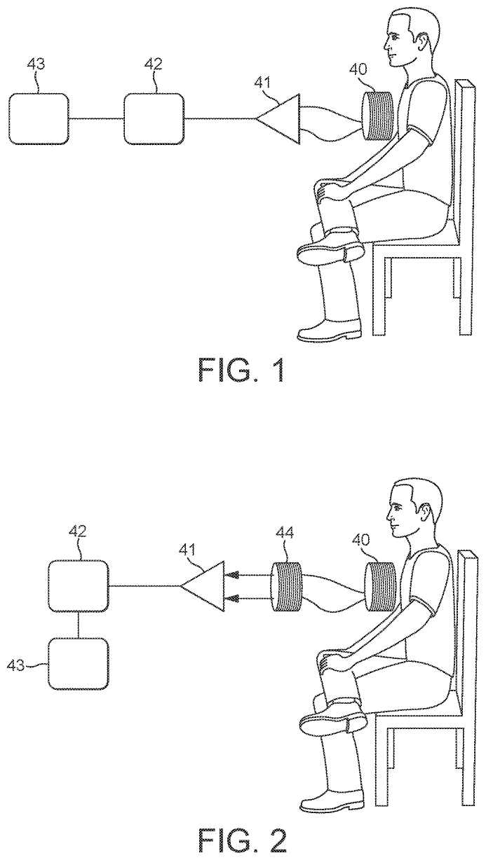

FIG. 1 shows schematically the use of an embodiment of the present invention for detecting the magnetic field of a subject's heart;

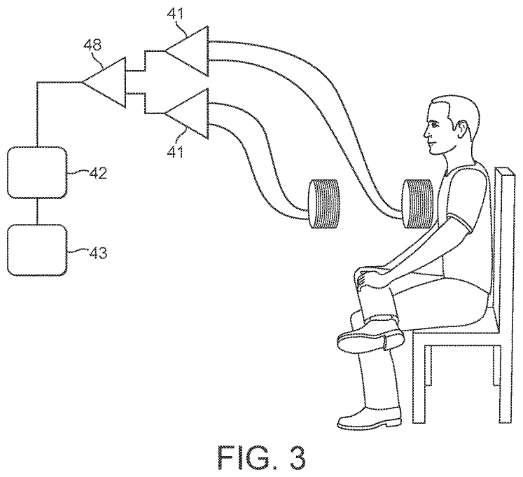

FIGS. 2-5 show further exemplary arrangements of the use of an embodiment of the present invention when detecting the magnetic field of a subject's heart;

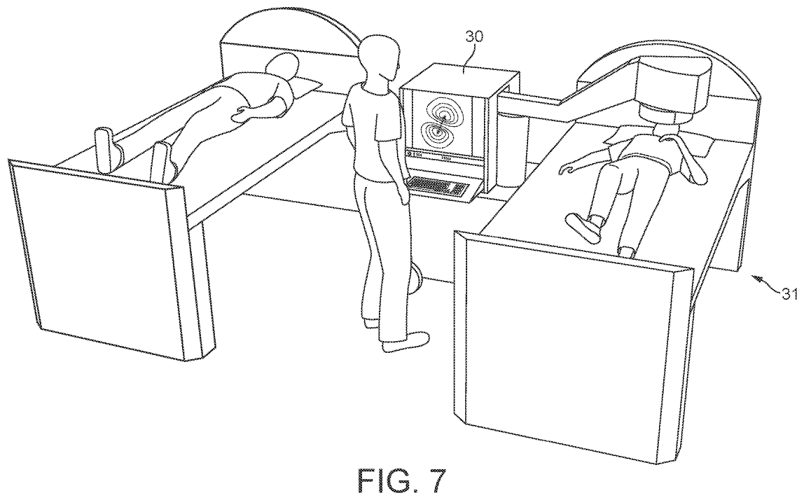

FIG. 6A shows schematically a coil arrangement in accordance with an embodiment of the present invention, and FIG. 6B shows schematically another coil arrangement in accordance with an embodiment of the present invention;

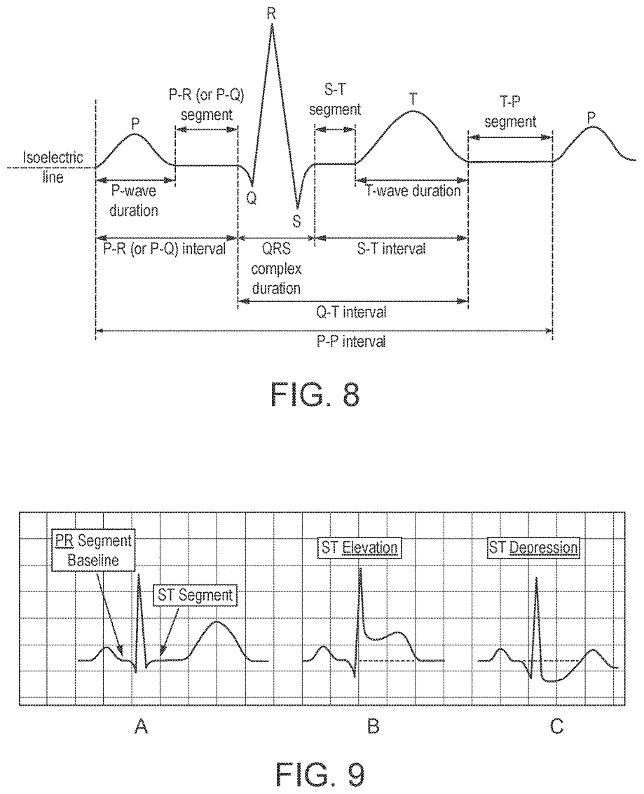

FIG. 7 shows a further exemplary arrangement of the use of an embodiment of the present invention when detecting the magnetic field of a subject's heart;

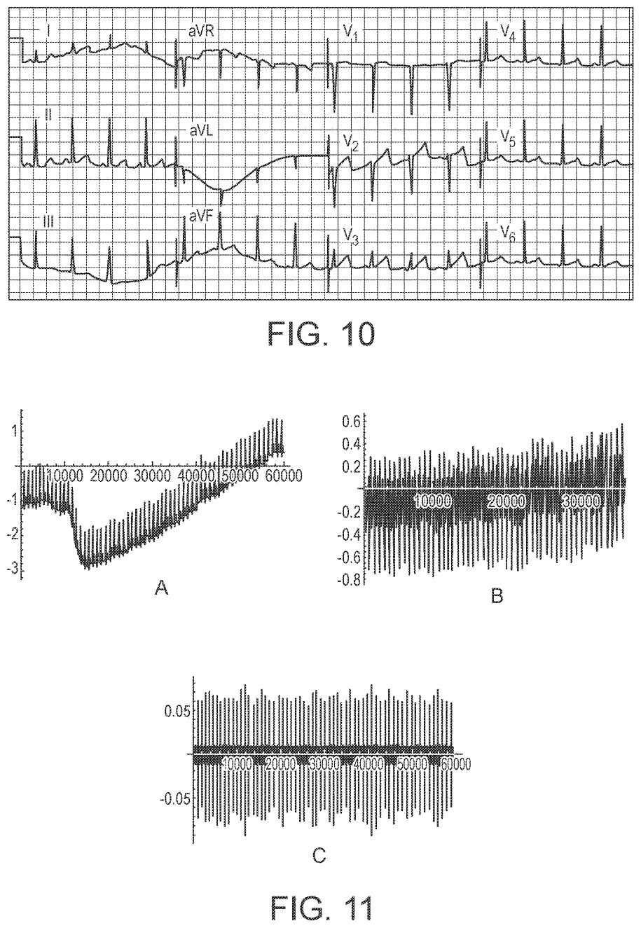

FIG. 8 shows a typical healthy ECG trace;

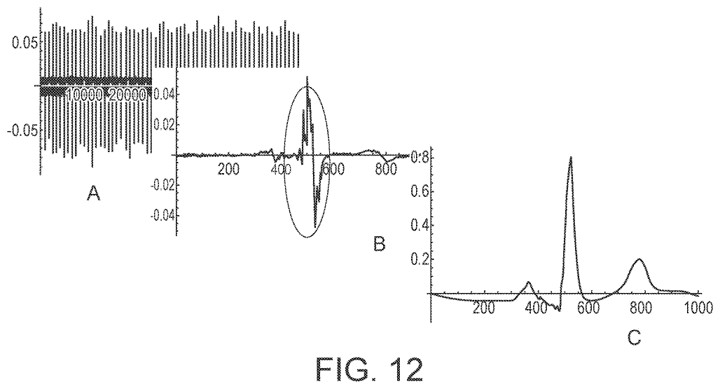

FIG. 9 shows three different ECG traces that are indicative of myocardial injury;

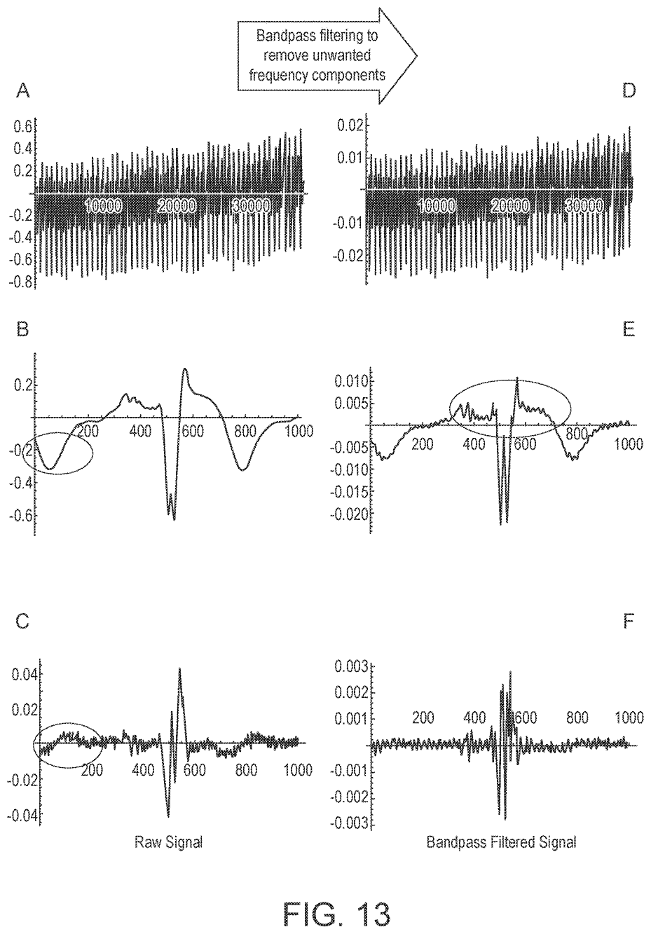

FIG. 10 shows ECG traces that exhibit baseline wander;

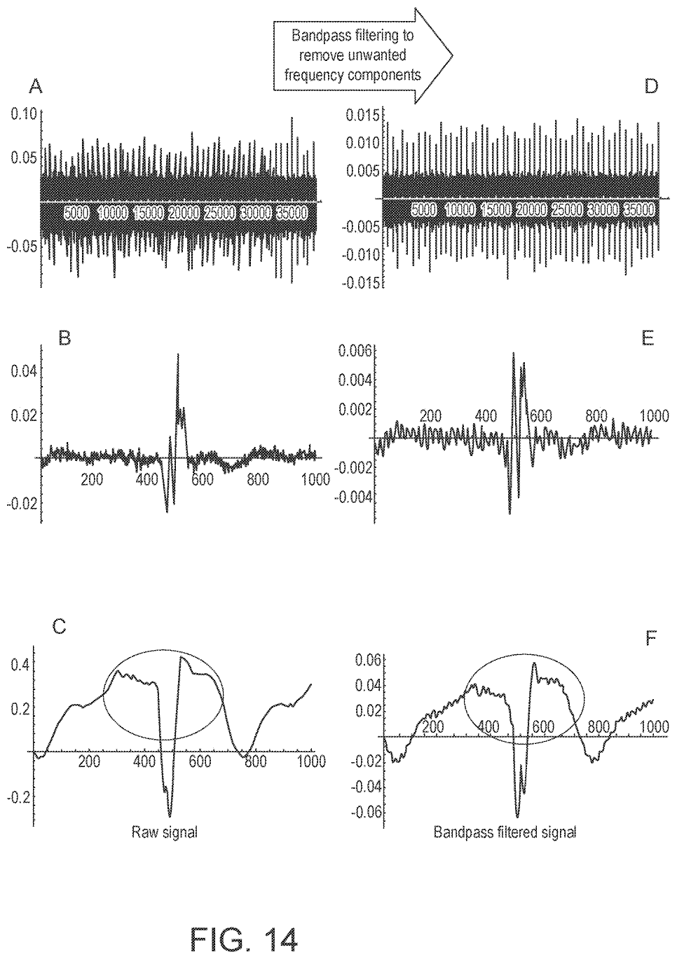

FIG. 11A shows raw ECG data exhibiting a large baseline shift; FIG. 11B shows the data of FIG. 11A filtered to remove baseline shifts; and FIG. 11C shows the derivative of the data of FIG. 11A without filtering;

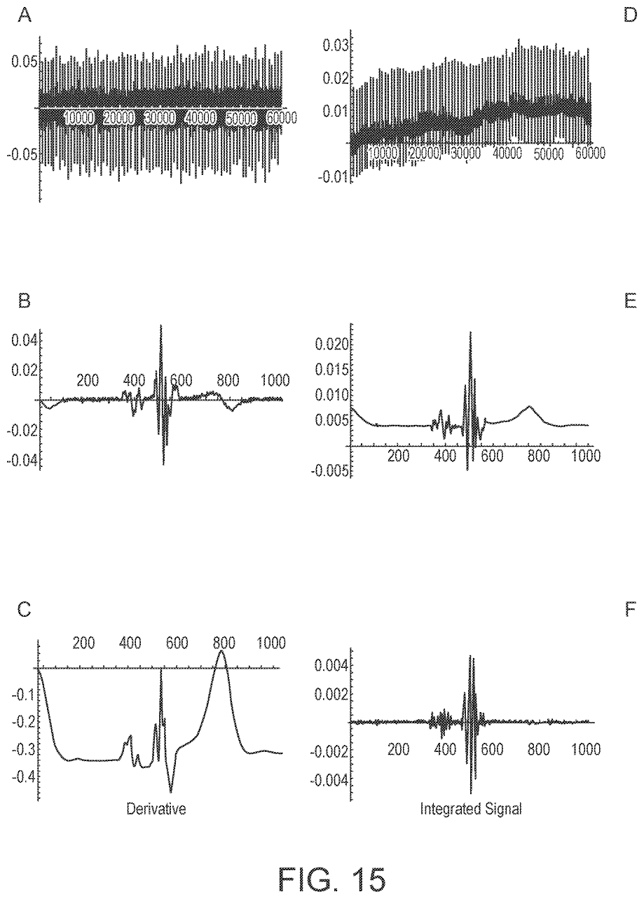

FIG. 12 illustrates the extraction of the average heartbeat from the raw data of FIG. 11 and its integration to show the “normal” time domain view;

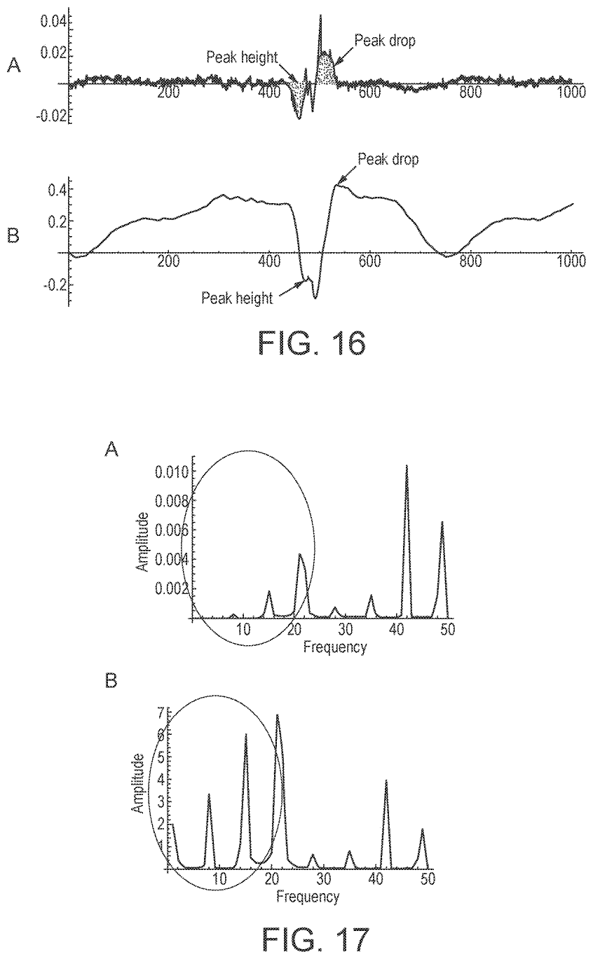

FIG. 13 shows data for a patient with Myocardial infarct;

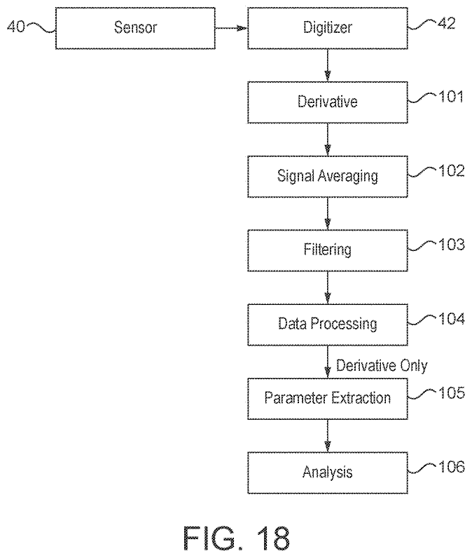

FIG. 14 shows data for the same patient with Myocardial infarct where the signal is processed in the derivative;

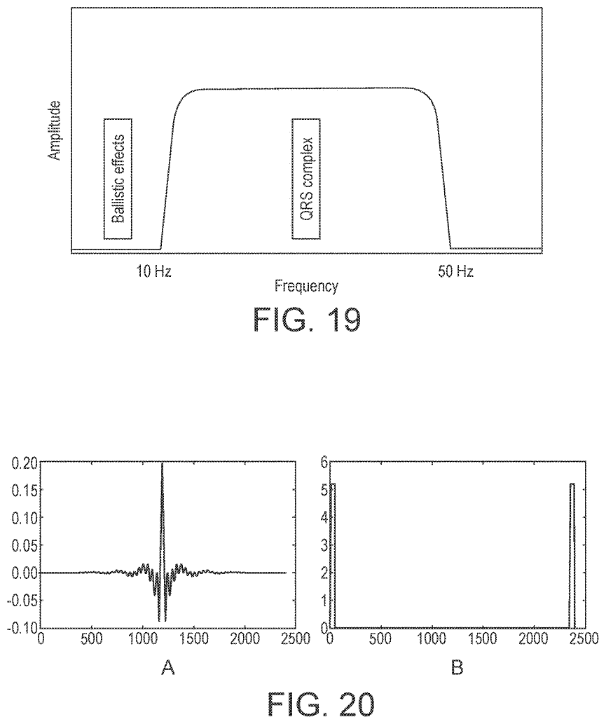

FIG. 15 shows data for another patient with Myocardial infarct where the signal is processed in the derivative;

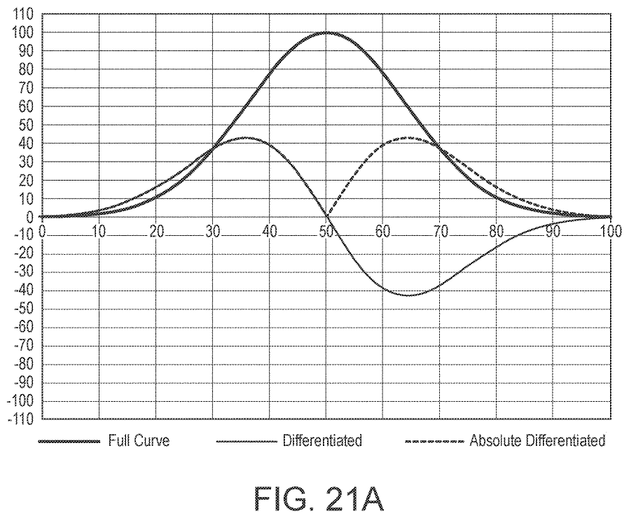

FIG. 16 shows data where the signal is processed in the derivative;

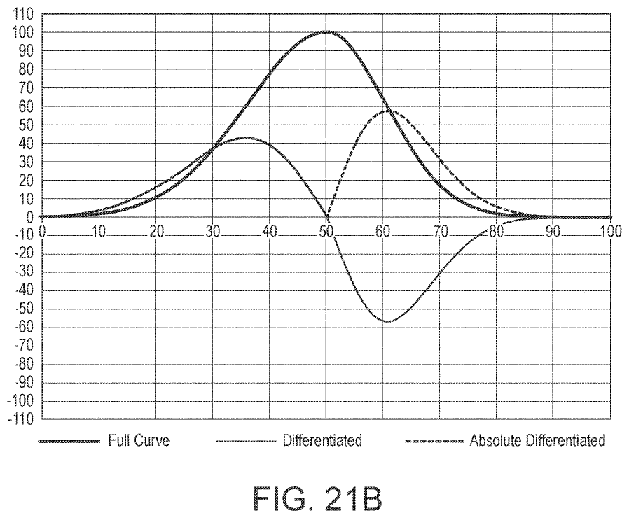

FIG. 17 shows the Fourier transform of the derivative and the integrated (“normal”) signal;

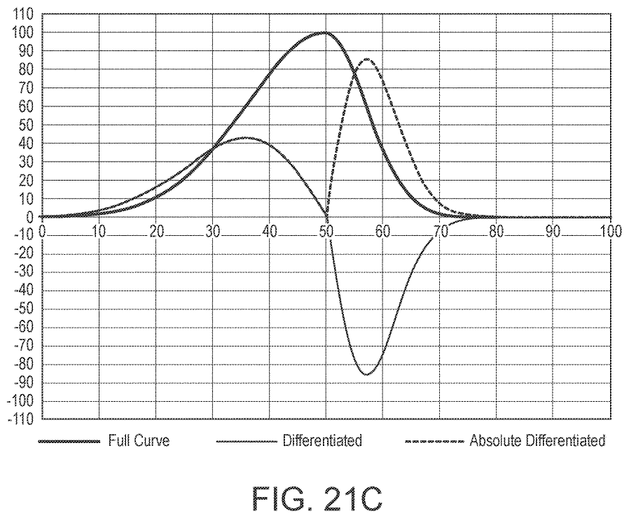

FIG. 18 illustrates a process in accordance with an embodiment of the present invention;

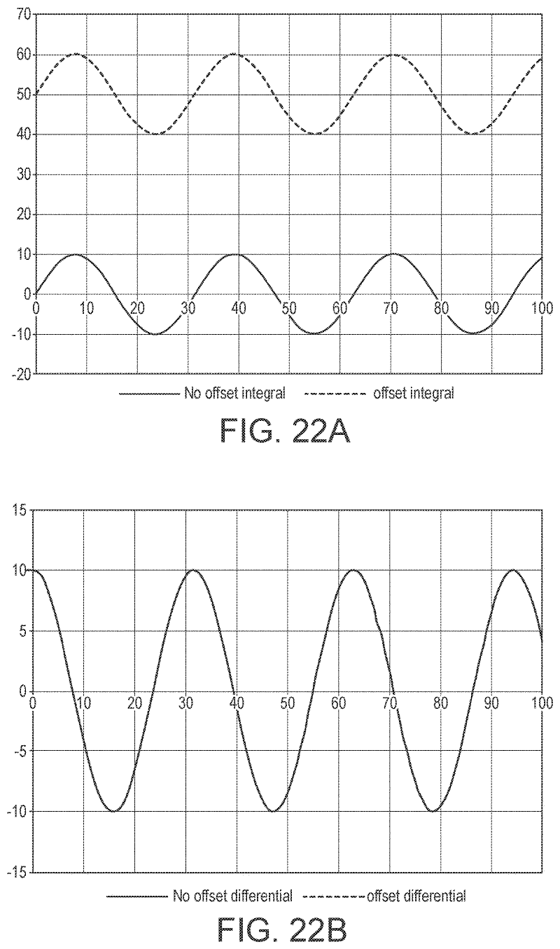

FIG. 19 illustrates an ideal band-pass filter in the frequency domain;

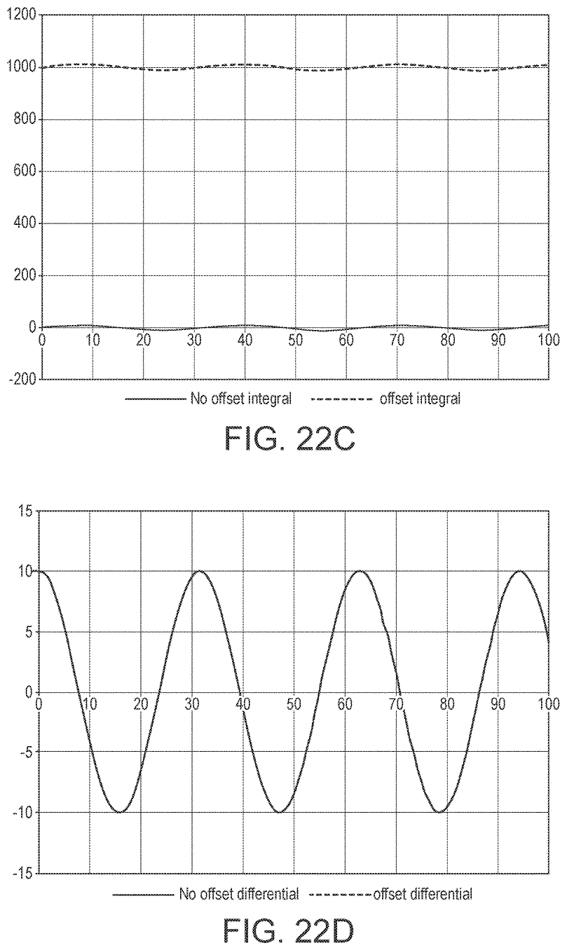

FIG. 20A shows a filter kernel formed from the difference between two windowed-sinc filters with cut-off frequencies at 8 Hz and 45 Hz, and M=2400, and

FIG. 20B shows the frequency response of the filter; and

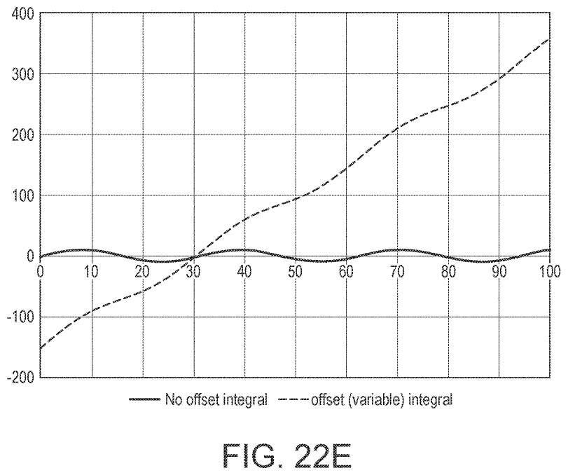

FIGS. 21A-C show various arbitrary time domain ECG or MCG signals in the form of Gaussian peaks with the same centres and amplitudes but different FWHMs for each half, together with their corresponding time derivative signals; and

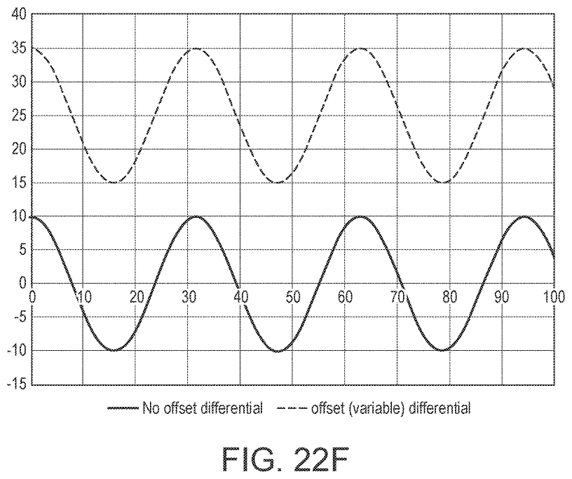

FIGS. 22A-F show various arbitrary time domain ECG or MCG signals in the form of sine waves with the same phase and amplitudes but different offsets together with their corresponding time derivative signals.

Like reference numerals are used for like components where appropriate in the Figures.

FIG. 1 shows schematically the basic arrangement of a preferred embodiment of a magnetometer system that may be operated in accordance with the present invention. This magnetometer system is specifically intended for use as a cardiac magnetometer (for use to detect the magnetic field of a subject's heart). However, the same magnetometer design can be used to detect the magnetic field produced by other body regions, for example for detecting and diagnosing bladder conditions, pre-term labour, foetal abnormalities and for magnetoencephalography. Thus, although the present embodiment is described with particular reference to cardio-magnetometry, it should be noted that the present embodiment (and the present invention) extends to other medical uses as well.

The magnetometer system comprises a detector 40 coupled to a detection circuit 41 that may contain a number of components. The detector 40 may be an induction coil 40.

The detection circuit 41 may comprise a low impedance pre-amplifier, such as a microphone amplifier, that is connected to the coil 40, a low pass filter, e.g. with a frequency cut off of 250 Hz, and a notch filter to remove line noise (e.g. 50 or 60 Hz and harmonics).

The current output from the coil 40 is processed and converted to a voltage by the detection circuit 41 and provided to an analogue to digital converter (ADC) 42 which digitises the analogue signal from the coil 40 and provides it to a data acquisition system 43.

A biological signal that is correlated to the heartbeat, e.g. an ECG or Pulse-Ox trigger from the test subject may be used as a detection trigger for the digital signal acquisition, and the digitised signal over a number of trigger pulses is then binned into appropriate signal bins, and the signal bins overlaid or averaged, by the data acquisition unit 43. Other arrangements would, however, be possible.

The coil 40 and detection circuit 41 may be arranged such that the coil 40 and the preamplifier of the detection circuit 41 are arranged together in a sensor head or probe which is then joined by a wire to a processing circuit that comprises the remaining components of the detection circuit 41. Connecting the sensor head (probe) and the processing circuit by wire allows the processing circuit to be spaced from the sensor head (probe) in use.

With this magnetometer, the sensor head (probe) will be used as a magnetic probe by placing it in the vicinity of the magnetic fields of interest.

FIG. 2 shows an improvement over the FIG. 1 arrangement, which uses in particular the technique of gradient subtraction to try to compensate for background noise. (Other techniques could, however, be used). In this case, an inverse coil 44 is used to attempt to subtract the effect of the background noise magnetic field from the signal detected by the probe coil 40. The inverse coil 44 will, as is known in the art, be equally sensitive to any background magnetic field, but only weakly sensitive to the subject's magnetic field. The inverse coil 44 can be accurately matched to the pickup coil 40 by, for example, using a movable laminated core to tune the performance of the inverse coil to that of the pickup coil 40.

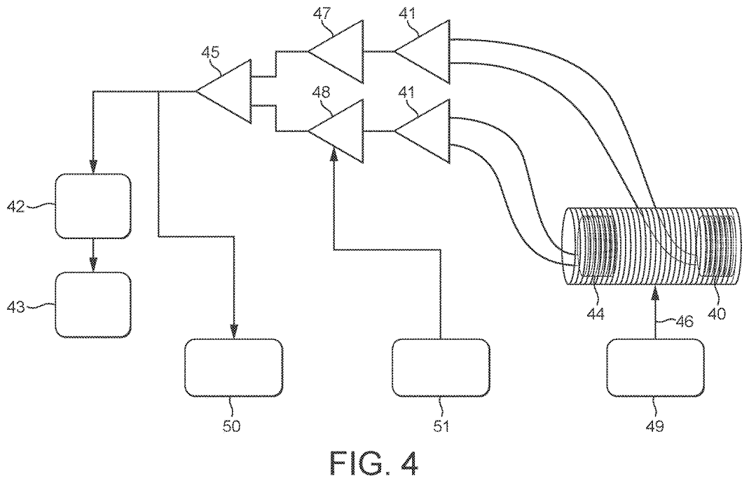

FIG. 3 shows an alternative gradient subtraction arrangement. In this case, both coils 40, 44 have the same orientation, but their respective signals are subtracted using a differential amplifier 45. Again, the best operation is achieved by accurately matching the coils and the performance of the detection circuits 41. Again, a movable laminated core can be used to tune the performance of one coil to match the performance of the other.

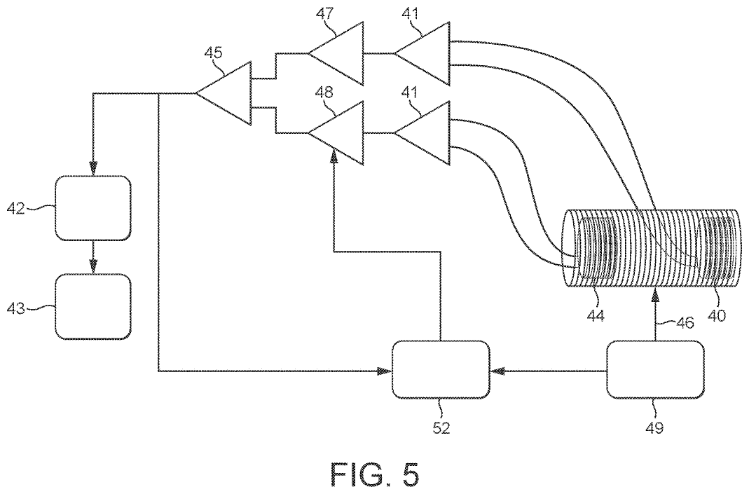

FIG. 4 shows a further preferred arrangement. This circuit operates on the same principle as the arrangement of FIG. 3, but uses a more sophisticated method of field cancellation, and passive coil matching. In particular, a known global magnetic field 44 is introduced to both coils 40, 44 to try to remove background magnetic field interference.

In this circuit, the outputs from the detection circuits 41 are passed through respective amplifiers 47, 48, respectively, before being provided to the differential amplifier 45. At least one of the amplifiers 47, 48 is tuneable. In use, a known global field 46, such as 50 or 60 Hz (and harmonics) line noise, or a signal, such as a 1 kHz signal, applied by a signal generator 49, is introduced to both coils 40, 44. The presence of a signal on this frequency on the output of the differential amplifier 45, which can be observed, for example, using an oscilloscope 50, will then indicate that the coils 40, 44 are not matched. An amplifier control 51 can then be used to tune the tuneable voltage controlled amplifier 48 to eliminate the global noise on the output of the differential amplifier 45 thereby matching the outputs from the two coils appropriately.

Most preferably in this arrangement, a known global field of 1 kHz or so is applied to both coils, so as to achieve the appropriate coil matching for the gradient subtraction, but also a filter to remove 50 or 60 Hz (and harmonics) noise is applied to the output signal.

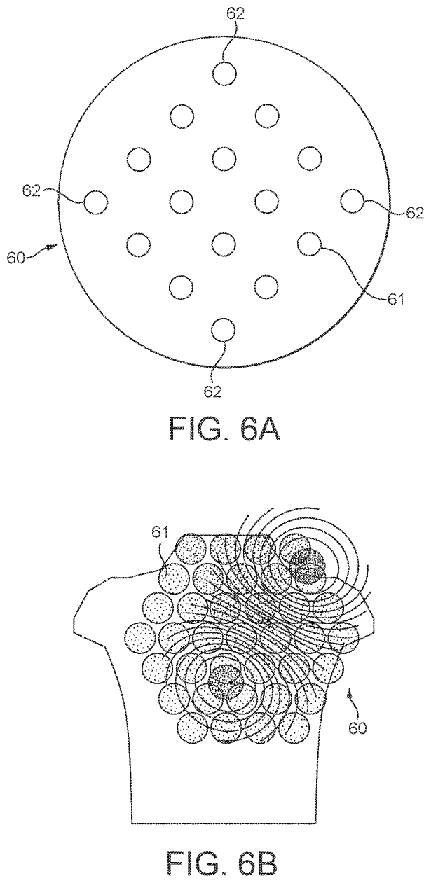

FIG. 5 shows a further variation on the FIG. 4 arrangement, but in this case using active coil matching. Thus, in this arrangement, the outputs of the coils 40, 44 are again channelled to appropriate detection circuits 41, and then to respective amplifiers 47, 48, at least one of which is tuneable. However, the tuneable amplifier 48 is tuned in this arrangement to remove the common mode noise using a lock in amplifier 52 or similar voltage controller that is appropriately coupled to the output from the differential amplifier 45 and the signal generator 49.

The above embodiments of the present invention show arrangements in which there is a single pickup coil that may be used to detect the magnetic field of the subject's heart. In these arrangements, in order then to make a diagnostic scan of the magnetic fields generated by a subject's heart, the single pickup coil can be moved appropriately over the subject's chest to take readings at appropriate spatial positions over the subject's chest. The readings can then be collected and used to compile appropriate magnetic field scans of the subject's heart.

It would also be possible to arrange a plurality of coil and detection circuit arrangements, e.g. of the form shown in FIG. 1, in an array, and to then use such an array to take measurements of the magnetic field generated by a subject's heart. In this case, the array of coils could be used to take readings from plural positions over a subject's chest simultaneously, thereby, e.g., avoiding or reducing the need to take readings using the same coil at different positions over the subject's chest.

FIGS. 6A and 6B show suitable coil array arrangements that have an array 60 of 16 detection coils 61, which may be then placed over a subject's chest to measure the magnetic field of a subject's heart at 16 sampling positions over the subject's chest. FIG. 6A shows a regular rectangular array and FIG. 6B shows a regular hexagonal array. In these cases, each coil 61 of the array 60 should be configured as described above and connected to its own respective detection circuit (i.e. each individual coil 61 will be arranged and have a detection circuit connected to it as shown in FIG. 1). The output signals from the respective coils 61 can then be combined and used appropriately to generate a magnetic scan of the subject's heart.

Other array arrangements could be used, if desired, such as circular arrays, irregular arrays, etc.

More (or less) coils could be provided in the array, e.g. up to 500 coils, or more than 500 coils. For example, where it is desired to measure the magnetic field of a different region of a subject's body (i.e. other than the heart), then an increased number of coils may be provided so as to provide an appropriate number of sampling points and an appropriate spatial coverage for the region of the subject's body in question.

It would also be possible in this arrangement to use some of the coils 61 to detect the background magnetic field for the purposes of background noise subtraction, rather than for detecting the wanted field of the subject's heart. For example, the outer coils 62 of the array could be used as background field detectors, with the signals detected by those coils then being subtracted appropriately from the signals detected by the remaining coils of the array. Other arrangements for background noise subtraction would, of course, be possible.

It would also be possible to have multiple layers of arrays of the form shown in FIG. 6, if desired. In this case, there could, for example, be two such arrays, one on top of each other, with the array that is closer to the subject's chest being used to detect the magnetic field generated by the subject's heart, and the array that is further away being used for the purposes of background noise detection.

To measure the magnetic fields generated by the heart, the above arrangements can be used to compile magnetic field scans of a subject's heart by collecting magnetic field measurements at intervals over the subject's chest. False colour images, for example, can then be compiled for any section of the heartbeat, and the scans then used, for example by comparison with known reference images, to diagnose various cardiac conditions. Moreover this can be done for significantly lower costs both in terms of installation and on-going running costs, than existing cardiac magnetometry devices.

FIG. 7 shows an exemplary arrangement of the magnetometer as it is envisaged it may be used in a hospital, for example. The magnetometer 30 is a portable device that may be wheeled to a patient's bedside 31 where it is then used to take a scan of the patient's heart (e.g.). There is no need for any magnetic shielding, cryogenic cooling, etc. The magnetometer 30 can be used in the normal ward environment. (Magnetic shielding and/or cooling could, however, be provided if desired.)

As used herein, a magnetometer or other apparatus in a “magnetically shielded” environment may comprise a magnetometer or other apparatus that is either arranged in a specially designed room or enclosure. In such arrangements, both the subject being measured and the equipment doing the measuring are contained within the same shielded enclosure. By contrast, as used herein, a magnetometer or other apparatus in a “non-magnetically shielded” comprises a magnetometer or other apparatus for which no external piece or pieces of apparatus are used to protect the subject being measured, nor the equipment doing the measuring.

The magnetometer system can be used in an analogous manner to detect and analyse other medically useful magnetic fields produced by other regions of the body, such as the bladder, head, brain, a foetus, etc.

FIG. 8 shows a typical ECG trace and the conventional labelling of the typical elements present in the ECG trace. Similar elements also occur in the MCG trace and the correspondence between the two has led to researchers using the same labelling convention.

As shown in FIG. 8, the ECG trace comprises a repeating P-P interval comprising a so-called P-wave, followed by a P-R (or P-Q) segment (where the combination of the P-wave and the P-R (or P-Q) segment is referred to as the P-R (or P-Q) interval), followed by a QRS complex, followed by an S-T segment, followed by a T-wave (where the combination of the S-T segment and the T-wave is referred to as the S-T interval, and the combination of the QRS complex and the S-T interval is referred to as the Q-T interval), followed by a T-P segment. Each of the features within the ECG can have diagnostic importance.

The signal generated by the induction coil in the present embodiment will be the derivative of the magnetic field. However, rather than integrating the output signal over time as would normally be done, the “raw” derivative signal is instead used for data analysis, etc.

Accordingly, the use of an induction coil represents a particularly convenient arrangement for obtaining a signal corresponding to the time derivative of the magnetic field, since for example, there is no need to differentiate the signal.

However, the Applicants have also found that there are benefits, e.g. as described below, when using a detector whose output signal corresponds to the time varying magnetic field, e.g. by differentiating the output signal to obtain a signal corresponding to the time derivative of the time varying magnetic field.

Thus, in the present embodiment, cardiac signals are analysed using the derivative of the magnetic field, dB/dt, rather than using the magnetic field B, as is conventional. Cardiac signals can also be analysed using the derivative of the voltage, dV/dt, rather than using the voltage V, as is conventional.

Analysis of the signal in the derivative is beneficial since, amongst other things, signal processing algorithms for the ECG and MCG must solve two conflicting problems, namely to remove background wander and to preserve biological signals which may have vital diagnostic importance. The conflict arises because frequently background effects can themselves have a biological origin.

FIG. 9A again shows an example of a normal, healthy ECG trace. FIGS. 9B and 9C show examples of ECG traces that indicate myocardial injury, where the S-T baseline is elevated (FIG. 9B) or depressed (FIG. 9C) with respect to the PR baseline. The MCG exhibits similar behaviour in corresponding regions of the complex albeit in different areas of the chest. The information content of the MCG is also different, with the S-T region being even more sensitive in MCG than in the ECG.

On the other hand, movement of a subject's limbs can cause baseline wander. FIG. 10 shows typical examples of baseline wander.

As such, movement of a subject's limbs can create low frequency baseline wander in the ECG signal, while a small shift in the S-T segment of the ECG can indicate myocardial infarction.

Separating baseline wander from shifts in the S-T baseline is relatively straightforward for a trained physician, but is somewhat more difficult for automated algorithms. The same cannot be said for MCG, principally because MCG lacks the decades of understanding and analysis that underpin the analysis of ECG. However, for a signal processing algorithm the task of separating the two is very challenging.

In the present embodiment, baseline wander is removed from the signal by using the derivative signal. Baseline drift is very low in frequency and therefore the derivative (dV/dt) is very small (i.e. dt is very large when the frequency of the drift is so small), so that using the derivative can negate the presence of baseline wander in the analysis of the results.

FIG. 11 shows ECG plots from a healthy volunteer (PTB ECG database). FIG. 11A shows the raw data showing large baseline shifts, FIG. 11B shows this data filtered to remove baseline shifts, and FIG. 11C shows the derivative data without filtering. It will be appreciated that the derivative data shown in FIG. 11C does not show a baseline shift.

The use of the derivative signal can accordingly remove or reduce the need for filtering. This is beneficial since with filtering there is always a risk that “real” signal will be removed when removing noise. This is particularly the case where the baseline drift is itself a biological signal. This accordingly means that more of the “wanted” signal can be retained for further analysis.

In the present embodiment, the derivative data is acquired repeatedly, and signal averaging techniques are applied to the data to produce the average heartbeat. This process is illustrated by FIG. 12.

FIG. 12A again shows the raw data from the healthy volunteer as shown in FIG. 11C. As illustrated by FIG. 12B, in the present embodiment this data is averaged over plural repeating periods to determine the average heartbeat. The average heartbeat of FIG. 12B may be used for diagnostic purposes. As shown in FIG. 12C, the average (derivative) heartbeat may be integrated to determine the average integrated heartbeat.

FIG. 12 shows the process of resolving a signal using the derivative. It can be seen in FIG. 12B that some frequency components that can be seen in the derivative are not visible in the “normal” time domain view. It can be seen from FIG. 12C that the integrated signal loses high frequency information.

FIG. 13 shows ECG data for a patient with Myocardial infarct indicated by the presence of S-T segment baseline shift. FIG. 13B shows the average heartbeat after averaging the raw data of FIG. 13A, where an S-T segment baseline shift can be seen in the average heartbeat. FIG. 13C shows the derivative of the averaged heartbeat.

FIGS. 13D-F show corresponding data where bandpass filtering has been applied. The use of bandpass filtering reduces the S-T segment shift because low frequency components are suppressed. This can be seen most clearly in the derivative, i.e. by comparing FIG. 13C (no filtering) with FIG. 13F (with filtering).

It can be seen from FIG. 13C that higher noise is present in the derivative due to an enhanced sensitivity to high frequency components. Lower frequency components are suppressed in FIG. 13F. FIG. 13E shows alterations to the T-wave, and changes to the R-peak structure.

FIG. 14 shows data for the same patient as FIG. 13, where the signal is processed in the derivative and then integrated. FIG. 14A shows the raw derivative data, FIG. 14B shows the data of FIG. 14A after averaging, and FIG. 14C shows the integrated version of the averaged heartbeat of FIG. 14B. FIGS. 14D-F show corresponding data where filtering has been used.

As can be seen from FIG. 14, the need to filter the signal to remove low frequency drift is obviated and the baseline S-T shift is retained by processing the data in the derivative.

FIG. 14E shows that higher frequency components are suppressed when the sound is filtered (as expected). However, as seen in FIG. 14F, the baseline shift is retained, the R-peak structure is only slightly altered, and the T-wave structure is unaltered.

As such, the Applicants have recognised that the derivative is a useful tool because (i) high frequency information that has a diagnostic value is naturally present in the derivative; and (ii) no additional filtering is necessary to arrive at the average heartbeat.

In addition, the low frequency structure of the signal is the same even if bandpass filtering is applied.

FIG. 15 show shows corresponding data to FIG. 14 but from a different Myocardial Infarct patient with an S-T baseline shift. FIG. 15C shows a baseline shift. The band pass signal in FIG. 15E shows no baseline shift, and FIG. 15F shows that low frequency data is removed. This illustrates that the results are repeated.

FIG. 16 illustrates why it is possible to filter and retain the relevant information when processing the data in the derivative. As shown in FIG. 16A, in the derivative, important information relating to the baseline shift is effectively moved from the S-T region into the QRS complex. The region marked “peak height” determines the R-Wave peak height and the region marked “peak drop” determines the subsequent fall. If these two regions have a similar area then there is very little baseline shift.

This can be seen in FIG. 17, where the frequency components of the two signals are compared. FIG. 17 compares the Fourier transform of the derivative (FIG. 17A) with the integrated (“normal”) signal (FIG. 17B). There is considerably less low frequency information in the derivative. However, it can be seen from the analysis above that the information concerning the state of the heart is preserved.

It will be appreciated that the derivative naturally reduces the scale of low frequency information, shifting it to higher frequencies within the complex. This, in turn, allows for filtering to be applied without destroying the relevant information.

FIG. 18 shows a sequence of data processing steps in accordance with the present embodiment.

A sensor 40 and a digitiser 42 are used to obtain a digitised derivative signal 101. As discussed above, this may be done by using the “natural” signal from a sensor that is configured to output a derivative signal, or by differentiating the digitised signal output from a sensor that is configured to output a magnetic field B or voltage V signal.

The differentiation may be performed in any suitable manner. Where, for example, the digitised signal comprises a sequence of values,

V(t)=[V₁,V₂,V₃, . . . ,Vₙ],

and where the values Vᵢ, Vᵢ₊₁ are separated by a fixed time step δt, then the derivative may be approximated by:

$$\frac{dV}{dt} \approx [ \frac{{V}_{1} - {V}_{2}}{δ t} , \frac{{V}_{2} - {V}_{3}}{δ t} , \frac{{V}_{3} - {V}_{4}}{δ t} , … , \frac{{V}_{n - 1} - {V}_{n}}{δ t} ] .$$

The digitised derivative signal is then averaged 102 over plural periods. This involves using a trigger such as the ECG to determine the plural repeating periods of the signal. Data is taken from the target waveform in each of plural windows around each of plural triggers. Several subsequent windows are averaged to remove random noise.

The use of the derivative signal is beneficial for this triggering operation, because the trigger is normally defined by the shape of the wave, or by a threshold detection. In either case, the removal of low frequency baseline shifts can improve triggering. Signal averaged ECG normally uses a trigger point derived from the ECG as the averaging position. This is prone to errors arising from baseline shifts whereas trigger derived from the derivative is not.

Additional filtering 103 may be applied, e.g. to remove noise that cannot be removed by averaging. For example, the digitised time derivative signal or signals may be filtered using (i) a notch filter to remove power line noise; and/or (ii) a bandpass filter to remove environmental noise. The digitised time derivative signal or signals may be filtered to remove external magnetic noise, e.g. arising from power lines and other environmental noise sources such as elevators, air conditioners, nearby traffic, mechanical vibrations, etc.

The Applicants have found, in particular, that a bandpass filter having a passband around 8-45 Hz can be used to separate the MCG signal from the environmental noise and background noise. The filter is a band pass filter constructed as combination of a high pass filter (removing environmental noise <10 Hz), and a low pass filter (removing background noise >50 Hz).

FIG. 19 illustrates an ideal band-pass filter. An idealised filter is one that removes all frequency components above a given cutoff frequency, without affecting lower frequencies, and has linear phase response. All frequencies within the passband 10-50 Hz, are passed with unity amplitude, while all other frequencies are blocked. The passband is perfectly flat, the attenuation in the stopband is infinite, and the transition between the two is infinitely small. The filter's impulse response is a sinc function in the time domain, and its frequency response is a rectangular function. It is an “ideal” low-pass filter in the frequency sense, perfectly passing low frequencies, perfectly cutting high frequencies, and thus may be considered to be a “brick-wall” filter.

In the present embodiment, in order to approximate such an ideal filter, two windowed-sinc filters are combined to construct a band-pass filter that can separate the MCG signal from environmental noise and background noise. This allows for an efficient separation of the QRS-complex from the environmental noise and other background noise interferences, without phase distortions.

The filter is configured such that it removes all frequency components below a cut-off frequency f_{c1} and above a cut-off frequency f_{c2} without affecting frequencies in between. The filter is designed as the difference of two windowed-sinc filters whose cut-off frequencies are f_{c1} and f_{c2}. The filter is able to significantly reduce the impact of environmental noise on the MCG signal, specifically the depolarisation (QRS) section.

FIG. 20A shows the filter kernel and FIG. 20B shows the frequency response of the difference of two windowed-sinc filters with cut-off frequencies f_{c1}=0.0033 (8.0 Hz), f_{c2}=0.01875 (45.0 Hz) and M=2400. The filter acts as a band pass filter.

The filter can be applied in either the time domain or the frequency domain to effectively separate the repolarisation (QRS section) of the MCG signal from the BCG effects and background noise.

Returning now to FIG. 18, diagnostic information can be extracted in the derivative, e.g. after other noise sources have been identified and removed.

Thus, following any additional data processing 104, diagnostic parameter extraction 105 may be performed, and used for analysis 106.

Some examples of medically useful signals that may be analysed are (i) S-T baseline shifts (STEMI) e.g. S-T elevated myocardial infarction (on taking the derivative, this becomes the R-S signal height); and (ii) R-S transition rate, e.g. bundle branch block (on taking the derivative, this becomes the R-S signal height).

However, in general any of the signal features described herein may have diagnostic importance and may be used for analysis. On taking the derivative, parameters that depend on a rate become a height, and parameters where a transition produces level shifts becomes a measurement of area.

It will be appreciated that, in the present embodiment, the derivative is used for analysis. The derivative emphasises high frequency information and supresses low frequency information. High frequency information can be diagnostic on its own. In addition, the derivative removes background drift without the need for filtering. It also concentrates information relating to S-T transition level shifts in the R-S region. This is a higher frequency region and therefore this signal can be separated from lower frequency components.

It should be noted that when analysing the magnetic field in the derivative domain, the rate of change, gradient, or slope of a feature may be used. The gradient of a feature in the integral corresponds to the amplitude of a feature in the derivative. This can allow more detailed or accurate diagnostic information to be obtained.

For example, as illustrated by FIGS. 21A-C, in the time domain ECG (and in the time domain MCG) a signal feature such as the QRS complex may comprise one or more single peaks. FIG. 21A shows a symmetric signal feature, FIG. 21B shows a slightly asymmetrical signal feature and FIG. 21C shows a moderately asymmetric feature.

As can be seen e.g. by comparing FIGS. 21A and 21B, it can be challenging to determine (or accurately measure), e.g., a slight imbalance or asymmetry in the (e.g. QRS) peak, e.g. if one side of the peak falls faster or slower than it rises (or vice versa).

By contrast, when using the derivative (MCG or ECG) signal, a signal feature (e.g. QRS complex) comprises two peaks, one corresponding to the rising edge (e.g. “QR”) and one to the falling edge (e.g. the “RS”) of the time domain feature (e.g. QRS complex). This means that, when using the derivative domain, any difference (imbalance or asymmetry) as described above is much easier to detect, e.g. since the two peaks will have different shapes and/or amplitudes. The same is true for other peaks and signal features in the averaged time derivative signal or signals.

In addition, small fluctuations on large absolute values (e.g. signals with large offsets or DC biases) can more readily be seen in the derivative than the integral. This is because upwards or downwards trends (or gradients/slopes) can be seen as positive or negative features in the derivative. For a sufficiently offset (or biased) signal, all values may remain positive (or negative) despite small fluctuations making it difficult to establish a trend.

This is illustrated by FIGS. 22A-F. FIGS. 22A and 22C show arbitrary time domain signals with and without an offset. By contrast, FIGS. 22B and 22D show the same signals in the derivative, where it can be seen the effect of the offset has been removed.

As also illustrated by FIGS. 22A-D, a large absolute value with a small fluctuation (e.g. 1000±10) is no different from a small absolute value with the same fluctuation (e.g. 1±10) in the derivative as only the fluctuation (e.g. ±10) is observed (i.e. as a peak with −10 amplitude and a second peak with +10 amplitude). In the integral these fluctuations are 1% and 1000% respectively of the absolute signal value, and can make it hard to locate a threshold with variable data, particularly in the case of a large absolute value with a small fluctuation (e.g. 1000±10) as all values may be positive.

In addition, for datasets or signals with increasing (or decreasing) DC or low frequency offsets, small fluctuations can more readily seen in the derivative than the integral. This is illustrated by FIGS. 22E-F, where it can be seen that subtle changes can be picked up in the derivative even when the offset is not constant.

As such, using the derivative domain in the manner of various embodiments can make diagnostic measurements more resistant to offsets or (e.g. DC) biases, i.e. since only change is measured. This can make it easier to deal with situations, for example, where a threshold value is of interest and is required to be measured. In particular, this can address the situation where, for example, it is desired to determine the value or location of a change from a positive to negative value in the MCG signal, but where because of an offset or (e.g. DC) bias, all values of the signal are positive or negative.

It can be seen from above that the present invention provides an improved magnetometer system for medical use.

This is achieved, in the preferred embodiments of the present invention at least, by obtaining one or more signals corresponding to the time derivative of the time varying magnetic field of a region of a subject's body, averaging the signal or signals, and using the averaged signal or signals to analyse the magnetic field generated by the region of the subject's body.

1. A method of using a magnetometer system to analyse the magnetic field of a region of a subject's body, the method comprising:
obtaining one or more signals corresponding to the time derivative of the time varying magnetic field of a region of a subject's body;
averaging the time derivative signal or signals over plural periods; and
using the averaged time derivative signal or signals to analyse the magnetic field generated by the region of the subject's body.

2. The method of claim 1, wherein obtaining one or more signals corresponding to the time derivative of the time varying magnetic field of the region of the subject's body comprises:
using a detector to produce a signal having a time varying magnitude that corresponds to the time derivative of the time varying magnetic field of the region of the subject's body.

3. The method of claim 1, wherein obtaining one or more signals corresponding to the time derivative of the time varying magnetic field of the region of the subject's body comprises:
using a detector to produce a signal having a time varying magnitude that corresponds the time varying magnetic field of the region of the subject's body; and
differentiating the produced signal to obtain a signal corresponding to the time derivative of the time varying magnetic field of the region of the subject's body.

4. (canceled)

5. (canceled)

6. (canceled)

7. The method of claim 1, wherein the one or more obtained signals comprise one or more digitised signals, and the method comprises:
averaging the digitised time derivative signal or signals over plural periods; and
using the averaged digitised time derivative signal or signals to analyse the region of the subject's body.

8. The method of claim 1, wherein averaging the time derivative signal or signals over plural periods comprises:
using a trigger to identify each repeating period of the time derivative signal or signals; and
averaging the signal over the plural identified periods;
wherein the trigger is determined using a time derivative signal or signals.

9. The method of claim 1, further comprising filtering the time derivative signal or signals.

10. The method of claim 1, wherein using the averaged time derivative signal or signals comprises at least one of:
extracting one or more diagnostic parameters from the averaged time derivative signal or signals and using the averaged time derivative signal or signals without integrating.

11. (canceled)

12. The method of claim 1, wherein the region of the subject's body comprises one of: the abdomen, bladder, heart, head, brain, chest, womb, one or more foetuses, or a muscle.

13. A magnetometer system for medical use, comprising:
one or more detectors for detecting the time varying magnetic field of a region of a subject's body;
detection circuitry configured to obtain from the one or more detectors one or more signals corresponding to the time derivative of a detected time varying magnetic field; and
averaging circuitry configured to average the time derivative signal or signals over plural periods;
wherein the magnetometer system is configured to use the averaged time derivative signal or signals to analyse the magnetic field generated by the region of the subject's body.

14. The system of claim 13, wherein the one or more detectors and the detection circuitry is configured to produce a signal or signals having a time varying magnitude that corresponds to the time derivative of the time varying magnetic field of the region of the subject's body.

15. The system of claim 13, wherein the one or more detectors and the detection circuitry is configured to produce a signal or signals having a time varying magnitude that corresponds the time varying magnetic field of the region of the subject's body; and
wherein the system further comprises processing circuitry configured to differentiate the magnetic field signal or signals to obtain the signal or signals corresponding to the time derivative of the time varying magnetic field of the region of the subject's body.

16. A electrocardiography system for medical use, comprising:
one or more detectors for detecting the time varying electric potential of a region of a subject's body;
detection circuitry configured to obtain from the one or more detectors one or more signals corresponding to the time derivative of a detected time varying electric potential; and
averaging circuitry configured to average the time derivative signal or signals over plural periods;
wherein the electrocardiography system is configured to use the averaged time derivative signal or signals to analyse the electric potential generated by the region of the subject's body.

17. The system of claim 16, wherein the one or more detectors and the detection circuitry is configured to produce a signal or signals having a time varying magnitude that corresponds to the time derivative of the time varying electric potential of the region of the subject's body.

18. The system of claim 16, wherein the one or more detectors and the detection circuitry is configured to produce a signal or signals having a time varying magnitude that corresponds to the time varying electric potential of the region of the subject's body; and
wherein the system further comprises processing circuitry configured to differentiate the electric potential signal or signals to obtain the signal or signals corresponding to the time derivative of the time varying electric potential of the region of the subject's body.

19. The system of claim 13, wherein:
the one or more obtained signals comprise one or more digitised signals;
the averaging circuitry is configured to average the digitised time derivative signal or signals over plural periods; and
the system is configured to use the averaged digitised time derivative signal or signals to analyse the region of the subject's body.

20. The system of claim 13, wherein the averaging circuitry is configured to average the time derivative signal or signals over plural periods by:
using a trigger to identify each repeating period of the time derivative signal or signals; and
averaging the signal over the plural identified periods;
wherein the averaging circuitry is configured to determine the trigger using a time derivative signal or signals.

21. The system of claim 13, further comprising one or more filters configured to filter the time derivative signal or signals.

22. The system of claim 13, wherein the system is configured to analyse the averaged time derivative signal or signals by extracting one or more diagnostic parameters from the averaged time derivative signal or signals.

23. The system of claim 13, wherein the system is configured to analyse the averaged time derivative signal or signals without integrating the averaged time derivative signal or signals.

24. The system of claim 13, wherein the region of the subject's body comprises one of: the abdomen, bladder, heart, head, brain, chest, womb, one or more foetuses, or a muscle.
